(19) United States
(12) Patent Application Publication
(10) Pub. No.: US 2023/0329636 A1
(43) Pub. Date: Oct. 19, 2023

(54) Respiratory Feedback For Improved Exercise Performance

(71) Applicant: QUALCOMM Incorporated, San Diego, CA (US)

(72) Inventors: Katheryn Victoria RAMP, Oakland, CA (US); Daniel James GUEST, San Diego, CA (US); Wen CHEN, Shanghai (CN); Yujie DAI, Shanghai (CN)

(21) Appl. No.: 18/044,660

(22) PCT Filed: Dec. 2, 2020
(86) PCT No.: PCT/CN2020/133279

§ 371 Date: Mar. 9, 2023

(51) Int. Cl. A61B 5/00; A61B 5/08; G16H 20/30

(52) U.S. Cl. CPC A61B 5/486; A61B 5/0816; G16H 20/30

(57) ABSTRACT

Systems and methods executed by a processor of user equipment for ensuring a user receives scheduled notifications are disclosed. Various embodiments may include determining a current exercise performed by the user, determining a target breathing pattern appropriate for the current exercise performed by the user, monitoring a current breathing pattern of the user while performing the current exercise based on inputs from a respiratory sensor, determining differences between the target breathing pattern appropriate for the current exercise performed by the user and the current breathing pattern of the user, and providing information to the user regarding determined differences between the target breathing pattern appropriate for the current exercise performed by the user and the current breathing pattern of the user.

### BACKGROUND

Breathing properly can help reduce stress, control emotions, improve attention, and maximize the benefits of exercise by increasing oxygenated blood flow to the heart. This helps prevent injury (like hernias or spikes in blood pressure) and improve exercise efficiency to allow people to exercise more comfortably for longer periods of time. Novice exercisers want to learn how to exercise properly and most effectively for their individual level of ability, but are often deterred by the expense of a personal trainer or too intimidated to go to a gym. In addition to learning the exercises themselves, learning the proper breathing techniques for such exercises can be difficult. Also, intermediate and expert exercisers may track their progress with hand-written notes or fitness tracking applications, but such tracking techniques do not provide feedback during an exercise. While some exercisers use wearable heart rate monitors, heart rate measurements may not provide needed information about whether the exerciser is performing the exercise properly and may not be useful for non-aerobic exercises such as yoga. Even cameras or kinetic sensors that monitor user movements do not provide user feedback designed to help improve breathing during a wide variety of exercises. Proper breathing form may include targeting a breath-to-movement cadence (e.g. 2 steps to 1 breath while running) and practice breathing deeply from the diaphragm (e.g., so the rib cage expands in all directions vs. shallow chest breaths). Breathing from the diaphragm easily, is also generally a good indicator of proper muscular form.

### SUMMARY

Various aspects include methods and computing devices implementing the methods executed by a processor of user equipment for providing information regarding breathing patterns of a user during exercise. Various aspects may include determining a current exercise performed by the user, determining a target breathing pattern appropriate for the current exercise performed by the user, monitoring a current breathing pattern of the user while performing the current exercise based on inputs from a respiratory sensor, determining differences between the target breathing pattern appropriate for the current exercise performed by the user and the current breathing pattern of the user, and providing information to the user regarding determined differences between the target breathing pattern appropriate for the current exercise performed by the user and the current breathing pattern of the user.

Some aspects may include receiving a sensor input from an exercise sensor providing information regarding user body movements, wherein determining the current exercise is based on the sensor input received from the exercise senor. In some aspects, the target breathing pattern is based on the received sensor input from the exercise sensor indicating how the user is moving during the exercise. Some aspects may include receiving a sensor input from an exercise sensor providing exercise information regarding the current exercise, wherein the exercise sensor is associated with exercise equipment used by the user to perform the current exercise.

Some aspects may include receiving user body movement information from an exercise sensor indicating which of a first and second part of the current exercise the user is currently performing. In some aspects, the target breathing pattern may include a first breathing pattern associated with the first part of the current exercise and a second breathing pattern different from the first breathing pattern and associated with the second part of the current exercise, determining differences between the target breathing pattern appropriate for the current exercise performed by the user and the current breathing pattern of the user may include determining differences between the target breathing pattern appropriate for the first and second parts of the current exercise and the current breathing pattern of the user during the first and second parts of the current exercise, and the information provided to the user may include differences between the target breathing pattern appropriate for the first and second parts of the current exercise and the current breathing pattern of the user during the first and second parts of the current exercise.

Some aspects may include receiving a manual user input regarding at least one of the current exercise or the target breathing pattern, wherein determining the target breathing pattern is further based on the received manual user input. Some aspects may include receiving contextual information indicating a context in which the user is performing the current exercise, wherein determining the target breathing pattern is further based on the received contextual information. In some aspects, the target breathing pattern may be based on at least one of a user's body type, health goals, or experience level performing the current exercise.

Some aspects may include determining another target breathing pattern for the user to achieve in response to the current breathing pattern exceeding a normal breathing pattern threshold and providing additional information to the user regarding the other target breathing pattern. Some aspects may include activating the respiratory sensor, configured to monitor the current breathing pattern of the user while performing the current exercise, in response to determining the current exercise is being performed by the user. In some aspects, determining differences between the target breathing pattern appropriate for the current exercise performed by the user and the current breathing pattern of the user may include comparing the current breathing pattern of the user to at least one of a previously determined respiratory rate, rhythm, or quality of the user when the user performed the current exercise.

Some aspects may include activating an additional sensor in response to the current breathing pattern of the user exceeding a normal breathing pattern threshold. Some aspects may include determining a first extent of body movements by the user attributed to the determined current exercise apart from breathing, wherein the current breathing pattern of the user is associated with a second extent of body movement by the user attributed to breathing and distinct from the first extent of body movement.

In some aspects, the current breathing pattern of the user includes at least one of a rate, rhythm, or quality of respiratory movement. In some aspects, providing information to the user regarding the determined differences includes notifying the user through at least one of a visual, audible, or haptic alert. In some aspects, the current exercise is determined based on exercise equipment used by the user and the information regarding the determined differences between the target breathing pattern appropriate for the current exercise performed by the user and the current breathing pattern of the user is provided to the user through feedback from the exercise equipment.

Further aspects include a user equipment device including a processor configured with processor-executable instructions to perform operations of any of the methods summarized above. Further aspects include a non-transitory processor-readable storage medium having stored thereon processor-executable software instructions configured to cause a processor to perform operations of any of the methods summarized above. Further aspects include a processing device for use in a computing device and configured to perform operations of any of the methods summarized above.

### BRIEF DESCRIPTION OF THE DRAWINGS

The accompanying drawings, which are incorporated herein and constitute part of this specification, illustrate exemplary embodiments, and together with the general description given above and the detailed description given below, serve to explain the features of Various embodiments.

FIG. 1A is a schematic diagram illustrating user equipment working in conjunction with wearables for providing information regarding breathing patterns of a user during a resistance training exercise in accordance with various embodiments.

FIG. 1B is a schematic diagram illustrating user equipment working in conjunction with wearables for providing information regarding breathing patterns of a user performing yoga postures in accordance with various embodiments.

FIG. 1C is a schematic diagram illustrating user equipment working in conjunction with wearables for providing information regarding breathing patterns of a user running on a treadmill in accordance with various embodiments.

FIG. 1D is a schematic diagram illustrating user equipment working in conjunction with wearables for providing information regarding breathing patterns of a user on a computerized exercise bicycle in accordance with various embodiments.

FIG. 2 is a block diagram illustrating components of an example system in a package for use in a computing device in accordance with various embodiments.

FIG. 3 is a component block diagram of an example system configured for executed by a processor of user equipment for ensuring a user receives scheduled notifications.

FIGS. 4A, 4B, 4C, 4D, 4E, 4F, 4G, 4H, 4I, and/or 4J show process flow diagrams of example methods for providing information regarding breathing patterns of a user executed by a processor of user equipment according to various embodiments.

FIG. 5 is a component block diagram of a network computing device suitable for use with various embodiments.

FIG. 6 is a component block diagram of a wireless computing device suitable for use with various embodiments.

FIG. 7 is a component block diagram of an example of smart glasses suitable for use with various embodiments.

### DETAILED DESCRIPTION

Various aspects will be described in detail with reference to the accompanying drawings. Wherever possible, the same reference numbers will be used throughout the drawings to refer to the same or like parts. References made to particular examples and embodiments are for illustrative purposes and are not intended to limit the scope of the various aspects or the claims.

Various embodiments provide methods executed by a processor of a user equipment for providing information regarding breathing patterns of a user during exercise. Various embodiments may include determining a current exercise performed by the user, determining a target breathing pattern appropriate for the current exercise, and determining a current breathing pattern of the user while performing the current exercise based on inputs from a respiratory sensor. In addition, differences between the target breathing pattern and the current breathing pattern of the user may be determined so that information may be provided to the user regarding determined difference.

As used herein, the term “breathing pattern” refers to the respiratory rate, depth, timing, and consistency of breaths of a user. While exercising, a user may strive to achieve a target breathing pattern configured to provide greater benefits to the user. To achieve the target breathing pattern, the user may attempt to better control body movements involved in the exercise or may attempt to directly change their breathing to make their current breathing pattern match the target breathing pattern.

As used herein, the term “computing device” refers to an electronic device equipped with at least a processor, communication systems, and memory configured with a contact database. Also, as used herein the term “user equipment” refers to a particular computing device from which a user may receive notifications. Computing devices, including user equipment, may include any one or all of cellular telephones, smart-phones, portable computing devices, personal or mobile multi-media players, laptop computers, tablet computers, 2-in-1 laptop/table computers, smart-books, ultrabooks, palmtop computers, wireless electronic mail receivers, multimedia Internet-enabled cellular telephones, wearable devices including smart-watches, smart-glasses, smart-contact lenses, augmented/virtual reality devices, entertainment devices (e.g., wireless gaming controllers, music and video players, satellite radios, etc.), and similar electronic devices that include a memory, wireless communication components and a programmable processor. In various embodiments, computing devices may be configured with memory and/or storage. Additionally, computing devices referred to in various example embodiments may be coupled to or include wired or wireless communication capabilities implementing various embodiments, such as network transceiver(s) and antenna(s) configured to communicate with wireless communication networks.

As used herein, the term “smart” in conjunction with a device, refers to a device that includes a processor for automatic operation, for collecting and/or processing of data, and/or may be programmed to perform all or a portion of the operations described with regard to Various embodiments herein. For example, a smart-phone, smart-glasses, smart-contact lenses, smart-watch, smart-ring, smart-necklace, smart-cup, smart-straw, smart-appliances, etc.

The term “system on chip” (SOC) is used herein to refer to a single integrated circuit (IC) chip that contains multiple resources and/or processors integrated on a single substrate. A single SOC may contain circuitry for digital, analog, mixed-signal, and radio-frequency functions. A single SOC may also include any number of general purpose and/or specialized processors (digital signal processors, modem processors, video processors, etc.), memory blocks (e.g., ROM, RAM, Flash, etc.), and resources (e.g., timers, voltage regulators, oscillators, etc.). SOCs may also include software for controlling the integrated resources and processors, as well as for controlling peripheral devices.

The term “system in a package” (SIP) may be used herein to refer to a single module or package that contains multiple resources, computational units, cores and/or processors on two or more IC chips, substrates, or SOCs. For example, a SIP may include a single substrate on which multiple IC chips or semiconductor dies are stacked in a vertical configuration. Similarly, the SIP may include one or more multi-chip modules (MCMs) on which multiple ICs or semiconductor dies are packaged into a unifying substrate. A SIP may also include multiple independent SOCs coupled together via high speed communication circuitry and packaged in close proximity, such as on a single motherboard or in a single wireless device. The proximity of the SOCs facilitates high speed communications and the sharing of memory and resources.

Various embodiments may use one or more of a variety of sensors to detect user activity associated with one or more exercises, as well as the user's breathing patterns during the detected activity. In particular, the sensors may be included in one or more adhesive patches, smart clothing, inertial measurement units, sensor-equipped wearable computing devices (also referred to herein as a “wearables”), sensor-equipped exercise equipment, or other sensors or sensor-equipped computing devices, to measure activity associated with an exercise and/or breathing patterns. Wearables may include smart glasses, ear-pieces, other head-worn devices, necklaces, chest straps, watches, bracelets, other wrist-worn device, and/or smart rings. Input from the one or more sensors may be used to automatically monitor a user's current breathing pattern and determine a target breathing pattern based on a detected current exercise performed by the user. In addition, information may be provided to the user regarding determined differences between the target breathing pattern appropriate for the current exercise and the current breathing pattern of the user.

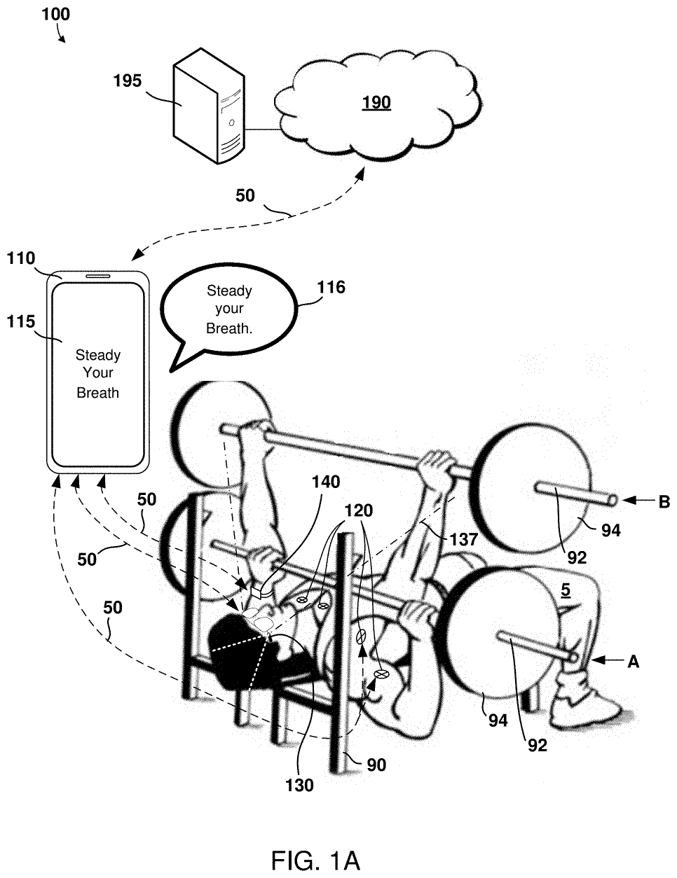

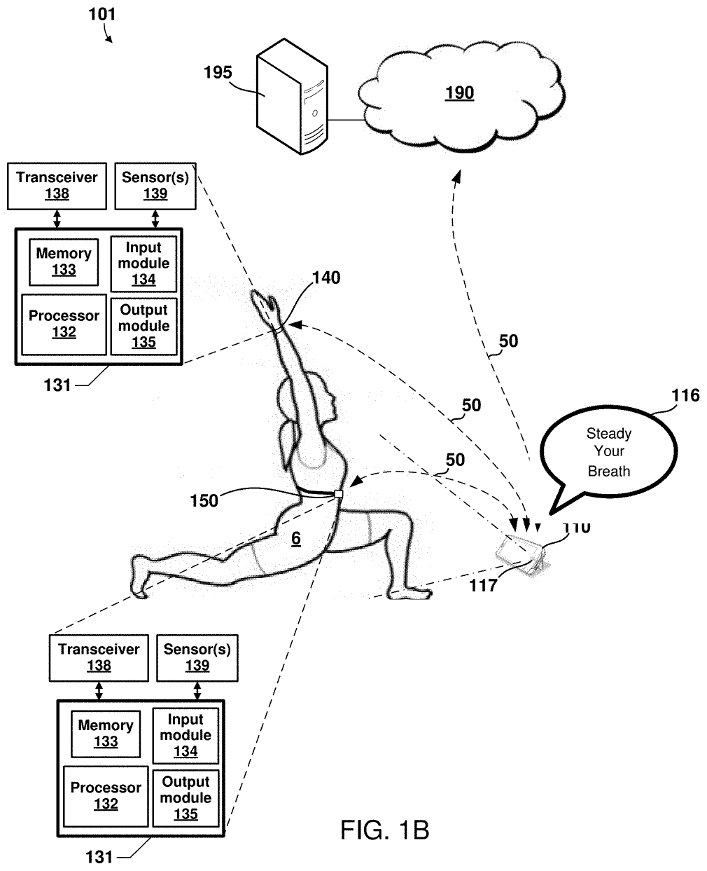

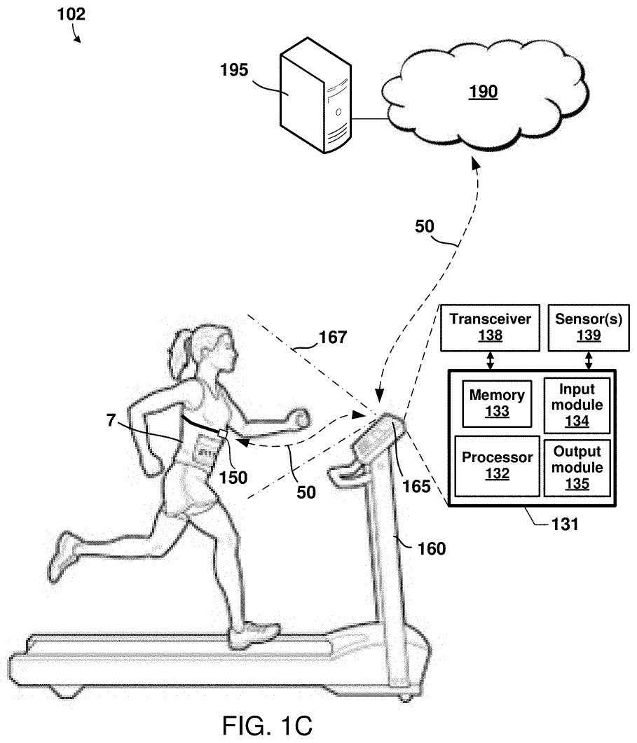

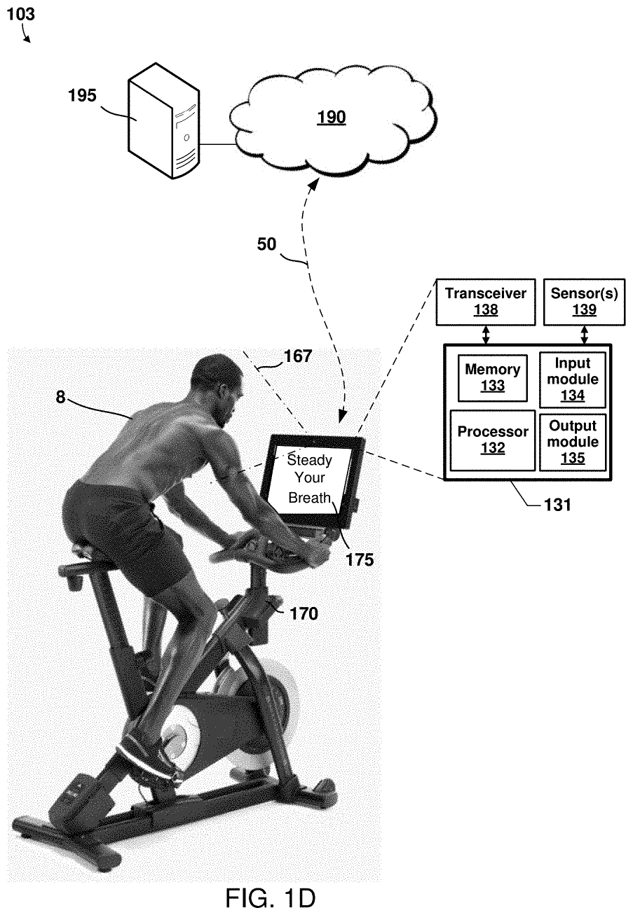

FIGS. 1A-1D illustrate several environments 100, 101, 102, 103 in which a user 5 is exercising, while various sensors are used to provide the user 5 feedback about the user's breathing as it relates to the exercise. In FIGS. 1A and 1B, the user 5, 6 has nearby user equipment 110, which may be configured to communicate through wireless connections 50 (e.g., Wi-Fi, Bluetooth, cellular, etc.) with remote computing devices (e.g., a server 195) through a wireless network 190, and may be supported by a wireless local area network router (not shown), such as a Wi-Fi wireless router, or a cellular network base station. In FIGS. 1C and 1D, the user 7, 8 is on exercise equipment 160, 170, which may be similarly configured to communicate through wireless connections 50 (e.g., Wi-Fi, Bluetooth, cellular, etc.) with the server 195 through a wireless network 190, and may be supported by a wireless local area network router (not shown), such as a Wi-Fi wireless router, or a cellular network base station.

FIG. 1A illustrates the environment 100 with the user 5 performing barbell bench presses (i.e., an exercise), while various sensors, such as adhesive patch sensors 120, smart-glasses 130, and/or a smart-watch 140 take measurements that are processed by user equipment 110 to determine differences between a target breathing pattern appropriate for the exercise and the current breathing pattern of the user 5, in accordance with various embodiments. Alternatively or additionally, the adhesive patch sensors 120, smart-glasses 130, and/or a smart-watch 140 may be a standalone processing unit with a display or other user interface (for providing feedback to the user) and include a processor configured to use the measurements to determine differences between a target breathing pattern appropriate for the exercise and the current breathing pattern of the user 5, in accordance with various embodiments.

To perform a conventional barbell bench press, the user 5 lays on a bench 90, holding a bar 92 carrying weights 94. The user 5 then raises the bar 92 carrying the weights 94 from a first position A near the user's chest to a second position B with arms outstretched away from the chest. The user 5 then lowers the bar 92 carrying the weights 94 back down to the chest and repeats the exercise for a number of repetitions. The amount of the weights 94 (i.e., how much they weigh collectively) is selected to limit the number of repetitions the user 5 can perform before fatiguing. Since conventional bench press equipment does not include sensors, various embodiments use additional equipment with sensors and a computing device, such as the user equipment 110, configured to provide information regarding breathing patterns of the user 5 during an exercise. The illustrated user equipment 110 is in the form of a cellular telephone (i.e., a smart-phone), although other forms of computing devices may be used (e.g., tablets, personal computers, exercise machines, wearables, smart-appliances, etc.).

The adhesive patch sensors 120 may be worn by the user 5 directly on the skin, over one or more major muscles used for the exercise (e.g., the chest, shoulders, neck, and/or the triceps). The adhesive patch sensors 120 may detect blood flow, electrical activity, sweat, motion of the underlying muscles, etc., and may operate battery-free (i.e., passive), powered by near-field communications (NFC), or include an onboard battery. In addition, through either the NFC or an onboard transceiver, the adhesive patch sensors 120 may communicate with the user equipment 110 or other computing device through wireless connections 50 (e.g., Wi-Fi, Bluetooth, cellular, etc.). The adhesive patch sensors 120 may include one or more processors configured to implement the methods of various embodiments described herein. In some embodiments, the adhesive patch sensors 120 may provide feedback to the user 5 through a speaker and/or haptic feedback device, thereby providing one or more user interfaces. Adhesive patch sensors 120 may operate as a standalone device or work in conjunction with other computing devices, including the user equipment 110, other wearables, and/or exercise equipment.

The smart-glasses 130 are a form of wearable that may include a built-in camera as well as heads-up display or augmented reality features on or near the front lenses. Like other sensor devices providing information to the user equipment 110, the smart-glasses 130 may include a processor or control unit configured to collect information from onboard sensors, such as a camera with a field of view 137. The camera may be used to capture images of exercise equipment and/or movements performed by the user to identify what exercise is being performed and/or what part of an exercise is being performed. For example, with the bar 92 within the field of view 137, images collected by the camera may be used to determine when the user is moving the bar 92 from position A to position B (concentric movement) or when the user is moving the bar 92 from position B to position A (eccentric movement). In addition, the smart-glasses 130 may include an electromyogram (e.g., in the arms of the frame), microphone, thermometer, and/or internal sensors, such an inertial measurement unit (IMU), proximity/motion sensor, light sensor, lidar, gas sensor, etc. The electromyogram may detect muscle movements associated with exercising. Microphones may detect breathing patterns from the mouth and/or nose. The thermometer may register the user's temperature and/or an ambient temperature around the user 5, which may provide contextual information relevant to determining a target breathing pattern for the user. The smart-glasses 130 may support wireless technologies like Bluetooth or Wi-Fi, enabling communications through a wireless connection 50 (e.g., a wireless communication link), such as to the user equipment 110 and/or other sensors (e.g., 120, 140). In addition, the smart-glasses 130 may control or retrieve data from the other sensors (e.g., 120, 140) and/or remote computing devices (110, 195). In some embodiments, the smart-glasses 130 may provide feedback to the user 5 through an augmented reality or heads-up display, a speaker, and/or haptic feedback device, thus serving as one or more user interfaces. The smart-glasses 130 may include one or more processors configured to implement the methods of various embodiments described herein. The smart-glasses 130 may operate as a standalone device or work in conjunction with other computing devices, including the user equipment 110, other wearables, and/or exercise equipment.

The smart-watch 140 is a form of wearable that may include an array of sensors, such as an electrical heart sensor to take ECG readings, an optical heart sensor to measure heart rate, a photoplethysmogram (PPG) sensor to estimate respiratory rate (e.g., detecting changes in blood volume), an accelerometer and/or gyroscope to track movement and rotation, a barometric altimeter to measure altitude, and an ambient light sensor to control the brightness of a display. The smart-watch 140 may include a processor or control unit configured to collect information from onboard sensors and support wireless technologies like Bluetooth or Wi-Fi, enabling communications through a wireless connection 50, such as to the user equipment 110 and/or other sensors (e.g., 120, 130). The smart-watch 140 may provide feedback to the user 5 through a display, a speaker, and/or haptic feedback device, and thus function as a user interface. In addition, the smart-watch 140 may control or retrieve data from the other sensors (e.g., 120, 130) and/or remote computing devices (110, 195). In some embodiments, the smart-watch 140 may operate with a mobile operating system for providing feedback to the user 5 through a watch-face display, vibrations, and/or sound. The smart-watch 140 may include one or more processors configured to implement the methods of various embodiments described herein. The smart-watch 140 may operate as a standalone device or work in conjunction with other computing devices, including the user equipment 110, other wearables, and/or exercise equipment.

In various embodiments, a processor of the user equipment 110, wearables, or other computing device may determine a current exercise performed by the user 5, such as through input received from sensors in one or more of the wearables 120, 130, 140. Based on the determined exercise, the processor may further determine a target breathing pattern appropriate for the current exercise performed by the user 5. The target breathing pattern may be based on at least one of a user's body type, health goals, or experience level performing the current exercise. In addition, further input received from sensors in one or more of the wearables 120, 130, 140 may be used by the processor to monitor a current breathing pattern of the user while performing the current exercise. The target breathing pattern may be based on received sensor inputs from one or more exercise sensors indicating how the user is moving during the current exercise. The current breathing pattern of the user may include at least one of a rate, rhythm, or quality of respiratory movement. For example, inputs received from sensors may be used to distinguish diaphragmatic breathing from thoracic breathing or vice versa. Similarly, different exercises may demand different breathing patterns. For example, running, cycling, and swimming may be more efficiently performed while maintaining a constant cadence or stroke rate, which may be detected from sensor inputs. Also, some exercises, such as swimming, may be more efficient if exhaling or inhaling is performed at a particular part of the stroke, which may similarly be detected from sensor inputs. The processor may then determine differences between the target breathing pattern and the current breathing pattern of the user. The processor may thus provide information to the user 5 regarding the determined differences between the target breathing pattern and the current breathing pattern. For example, the user equipment 110 may be configured to provide feedback to the user 5 through the display 115, such as an alert that may read, “Steady Your Breath,” or provide an audible feedback 116 using a speaker of the user equipment 110, such as an automated voice output saying, “Steady your breath,” “Breathe from your belly,” a customized message, and/or other message.

In some embodiments, the processor may also or additionally receive a sensor input from an exercise sensor, such as from exercise equipment being using during an exercise (e.g., treadmill, bicycle, rower, elliptical, etc.) providing information regarding user body movements useful in determining the current exercise. For example, by analyzing video images of what the user 5 is doing (e.g., captured by the smart-glasses 130), and/or other sensor inputs detecting movements, sounds, vibrations, or muscle activity, a processor of the user equipment 110 may not only determine a current exercise performed by the user (e.g., barbell bench presses, running, cycling, swimming, etc.), but also monitor a current breathing pattern of the user 5.

In some embodiments, the processor may receive user body movement information from an exercise sensor indicating how the user is moving during an exercise. Additionally or alternatively, the processor may receive user body movement information from an exercise sensor indicating which of a first and second part of the current exercise the user is currently performing. For example, the adhesive patch sensors 120, which may be placed on the skin near specific training muscles (i.e., the primary muscles used in a particular exercise) or other wearables, like the smart-watch 140, may provide the processor with input used to determine not only what exercise is being performed, but also the specific body movements that are being performed and/or the specific part of an exercise that is being performed. In addition, sensors placed on the diaphragm may be used to monitor the user's current breathing pattern during the exercise, including particular parts of the exercise. The processor may correlate measured diaphragm movements associated with current breathing patterns to muscle movements associated with individual parts of an exercise. Using yoga as example, a target breathing pattern may involve exhaling while stretching further into a pose (i.e., a first part of the current exercise) and then inhaling when releasing the pose (i.e., a second part of the current exercise). In this way, the determined target breathing pattern may include a first breathing pattern associated with a first part of the current exercise and a second breathing pattern, different from the first breathing pattern and associated with the second part of the current exercise. As another example, in resistance training, a first part of the exercise may involve lifting the weight (i.e., a pushing force), which should be performed while exhaling, while a second part of the exercise may involve holding the weight in an elevated position, which should be performed while inhaling, just before a lowering movement performed while holding one's breath. During a running, swimming, or cycling exercise, different parts of the movements may be associated with different parts of breathing.

In addition, the processor may determine differences between the target breathing patterns appropriate for the respective first and second parts of the current exercise and the current breathing patterns of the user during the respective first and second parts of the current exercise. Also, the processor may provide information to the user 5 that includes the differences between the target breathing patterns appropriate for the first and second parts of the current exercise and the current breathing patterns of the user during the first and second parts of the current exercise.

In some embodiments, in response to determining the current exercise is being performed by the user 5, the processor may activate a respiratory sensor (i.e., a sensor configured to measure a current breathing pattern or aspects of a current breathing pattern). For example, the processor may activate one or more sensors in the smart-glasses 130 configured to measure current breathing patterns. In this way, the respiratory sensor need not be active continuously, which may conserve power, but once activated be configured to monitor the current breathing pattern of the user while performing the current exercise.

The user equipment 110, adhesive patch sensors 120, smart-glasses 130, and/or a smart-watch 140 may include other or additional sensors, such as the camera, microphone, IMU, clocks, electromyograms, gas sensors, pressure sensor, proximity/motion sensor, light sensor, and thermometer. Although three wearables 120, 130, 140 are illustrated in FIG. 1A, various embodiments may include fewer or a greater number of wearables and/or remote computing devices, including one or more different types of wearables and/or computing device not shown in the environment 100.

The user equipment 110 may use conventional functions applied to the determination of what exercise is being performed, a target breathing pattern, or the current breathing pattern, such as a clock/timer that may provide a measure of how long movements or parts of a breathing pattern last, which may indicate what exercise is performed, what part of an exercise is being performed, and/or the user's current breathing pattern.

FIG. 1B illustrates an example 101 in which the user 6 is performing yoga (i.e., an exercise), while various sensors, such as a smart-watch 140, chest-strap sensor 150, and a camera 117 in the user equipment 110, provide information useful in determining the current exercise (i.e., the current yoga pose in the illustrated example), as well as measuring the current breathing pattern of the user 6. With this information, the user equipment 110 may determine differences between a target breathing pattern appropriate for the current yoga pose and the measured breathing pattern in accordance with various embodiments.

In accordance with various embodiments, a processor in the user equipment or other computing device may provide information to the user that may help the user properly coordinate breathing patterns with each movement of an exercise. In some embodiments, the user equipment 110 may provide information to the user regarding determined differences between a current breathing pattern and a target breathing pattern. The information may be provided to the user 6 through at least one of a visual, audible, or haptic alert. In FIG. 1B, the user equipment 110 is emitting verbal instructions (i.e., audible feedback 116) that instruct the user 6 to “Steady Your Breath.” This type of instruction may be helpful to remind the user to relax and coordinate her breathing with each movement.

In accordance with various embodiments, existing wrist-worn sensors, such as the smart-watch 140, may be improved in a multitude of ways to increase their value for various exercises. The smart-watch 140 or other wrist-worn sensor may be used for power measurements and/or breath tracking. Power measurements may measure levels of muscular exertion, such as during a difficult isometric yoga pose requiring the user maintain a static contraction of muscles without significant movement in the joints. Plus, using one wrist-worn sensor on each arm may greatly improve accuracy for detecting the type of exercise as well as the number of repetitions compared to using only a single wrist-worn sensor on one of the user's two arms.

The smart-watch 140 and the chest strap sensor 150 may each include a control unit 131, which may include various circuits and devices used to control operations thereof. In the example illustrated in FIG. 1B, the control unit 131 includes a processor 132, memory 133, an input module 134, and an output module 135. In addition, the control unit 131 may be coupled to a transceiver 138 for transmitting and/or receiving wireless communications and one or more sensors 139. In some embodiments, like the smart-watch 140, the chest-strap sensor 150 may provide feedback to the user 6 through a speaker and/or a haptic feedback device, which may be on the smart-watch 140, the chest strap sensor 150, or both, thereby providing one or more user interfaces.

FIG. 1C illustrates the environment 102 with the user 7 running on a treadmill 160 (i.e., performing an exercise), while various sensors, such as the chest-strap sensor 150 and sensors in the treadmill 160 (i.e., the exercise equipment) including a camera (shown as the camera imaging angle 167), provide information useful in determining differences between a target breathing pattern appropriate for the exercise and the current breathing pattern of the user 7, in accordance with various embodiments.

The treadmill 160 may similarly include a control unit 131 with a processor 132, memory 133, an input module 134, and an output module 135 that may render feedback information on a display 165 of the treadmill 160. In addition, the control unit 131 may be coupled to a transceiver 138 for transmitting and/or receiving wireless communications and one or more sensors 139. The treadmill 160 may provide feedback to the user 7 through a display, a speaker, and/or haptic feedback device (e.g., a vibrator on or in the handholds), thereby providing one or more user interfaces.

In some embodiments, sensor-equipped exercise equipment may include a treadmill, elliptical machine, exercise bike, and/or rower. In this way, a processor may receive a sensor input from an exercise sensor providing exercise information regarding the current exercise, wherein the exercise sensor is associated with exercise equipment used by the user to perform the current exercise.

FIG. 1D illustrates the environment 103 with the user 8 riding an exercise bicycle 170 (i.e., performing an exercise), while various sensors in the exercise bicycle 170, including a camera (shown as the camera imaging angle 167), provide information useful in determining differences between a target breathing pattern appropriate for the exercise and the current breathing pattern of the user 6, in accordance with various embodiments.

The exercise bicycle 170 may similarly include a control unit 131 with a processor 132, memory 133, an input module 134, and an output module 135 coupled to a display 175 on which feedback may be displayed. In addition, the control unit 131 may be coupled to a transceiver 138 for transmitting and/or receiving wireless communications and one or more sensors 139. The exercise bicycle 170 may provide feedback to the user 8 through a display, a speaker, and/or haptic feedback device (e.g., a vibrator on or in the handle bars), thereby providing one or more user interfaces.

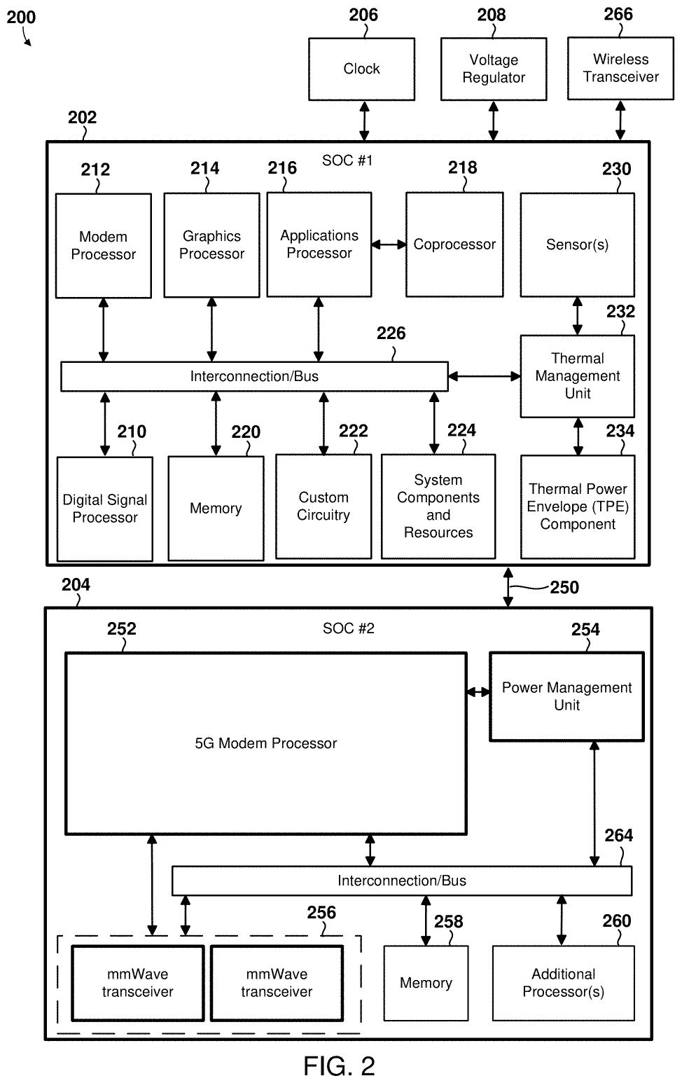

Various embodiments may be implemented in various types of user equipment, computing devices, and control units in other devices using a number of single processor and multiprocessor computer systems, including a system-on-chip (SOC) or system in a package (SIP). FIG. 2 illustrates an example computing system or SIP 200 architecture that may be used in a computing device, such as the user equipment (e.g., 110) and/or one or more of the wearables (e.g., 130, 150, 170, etc.) implementing Various embodiments.

With reference to FIGS. 1A-2, the illustrated example SIP 200 includes a two SOCs 202, 204, a clock 206, a voltage regulator 208, and a wireless transceiver 266. In some embodiments, the first SOC 202 operates as central processing unit (CPU) of the wireless device that carries out the instructions of software application programs by performing the arithmetic, logical, control and input/output (I/O) operations specified by the instructions. In some embodiments, the second SOC 204 may operate as a specialized processing unit. For example, the second SOC 204 may operate as a specialized 5G processing unit responsible for managing high volume, high speed (e.g., 5 Gbps, etc.), and/or very high frequency short wave length (e.g., 28 GHz mmWave spectrum, etc.) communications.

The first SOC 202 may include a digital signal processor (DSP) 210, a modem processor 212, a graphics processor 214, an application processor 216, one or more coprocessors 218 (e.g., vector co-processor) connected to one or more of the processors, memory 220, custom circuitry 222, system components and resources 224, an interconnection/bus module 226, one or more sensors 230 (e.g., thermal sensors, motion sensors, proximity sensors, a multimeter, etc.), a thermal management unit 232, and a thermal power envelope (TPE) component 234. The second SOC 204 may include a 5G modem processor 252, a power management unit 254, an interconnection/bus module 264, a plurality of mmWave transceivers 256, memory 258, and various additional processors 260, such as an applications processor, packet processor, etc.

Each processor 210, 212, 214, 216, 218, 252, 260 may include one or more cores, and each processor/core may perform operations independent of the other processors/cores. For example, the first SOC 202 may include a processor that executes a first type of operating system (e.g., FreeBSD, LINUX, OS X, etc.) and a processor that executes a second type of operating system (e.g., MICROSOFT WINDOWS 10). In addition, any or all of the processors 210, 212, 214, 216, 218, 252, 260 may be included as part of a processor cluster architecture (e.g., a synchronous processor cluster architecture, an asynchronous or heterogeneous processor cluster architecture, etc.).

The first and second SOC 202, 204 may include various system components, resources and custom circuitry for managing sensor data, analog-to-digital conversions, wireless data transmissions, and for performing other specialized operations, such as decoding data packets and processing encoded audio and video signals for rendering in a web browser. For example, the system components and resources 224 of the first SOC 202 may include power amplifiers, voltage regulators, oscillators, phase-locked loops, peripheral bridges, data controllers, memory controllers, system controllers, access ports, timers, and other similar components used to support the processors and software clients running on a wireless device. The system components and resources 224 and/or custom circuitry 222 may also include circuitry to interface with peripheral devices, such as cameras, electronic displays, wireless communication devices, external memory chips, etc.

The first and second SOC 202, 204 may communicate via interconnection/bus module 250. The various processors 210, 212, 214, 216, 218, may be interconnected to one or more memory elements 220, system components and resources 224, and custom circuitry 222, and a thermal management unit 232 via an interconnection/bus module 226. Similarly, the processor 252 may be interconnected to the power management unit 254, the mmWave transceivers 256, memory 258, and various additional processors 260 via the interconnection/bus module 264. The interconnection/bus module 226, 250, 264 may include an array of reconfigurable logic gates and/or implement a bus architecture (e.g., CoreConnect, AMBA, etc.). Communications may be provided by advanced interconnects, such as high-performance networks-on chip (NoCs).

The first and/or second SOCs 202, 204 may further include an input/output module (not illustrated) for communicating with resources external to the SOC, such as a clock 206 and a voltage regulator 208. Resources external to the SOC (e.g., clock 206, voltage regulator 208) may be shared by two or more of the internal SOC processors/cores.

In addition to the example SIP 200 discussed above, various embodiments may be implemented in a wide variety of computing systems, which may include a single processor, multiple processors, multicore processors, or any combination thereof.

In some embodiments, only one SOC (e.g., 132, 202) may be used in a less capable computing device, such as wearables (e.g., 130, 150, 170, etc.) that are configured to provide sensor information to a more capable user equipment, such as a smart phone (e.g., UE 110). In such embodiments, communication capabilities of the wearable (e.g., 130, 150, 170, etc.) may be limited to a short-range communication link, such as Bluetooth or Wi-Fi, in which case the 5G capable SOC 204 may not be included in the processing system of the wearable.

As used herein, the terms “component,” “system,” “unit,” “module,” and the like include a computer-related entity, such as, but not limited to, hardware, firmware, a combination of hardware and software, software, or software in execution, which are configured to perform particular operations or functions. For example, a component may be, but is not limited to, a process running on a processor, a processor, an object, an executable, a thread of execution, a program, and/or a computer. By way of illustration, both an application running on a communication device and the communication device may be referred to as a component. One or more components may reside within a process and/or thread of execution and a component may be localized on one processor or core and/or distributed between two or more processors or cores. In addition, these components may execute from various non-transitory computer readable media having various instructions and/or data structures stored thereon. Components may communicate by way of local and/or remote processes, function or procedure calls, electronic signals, data packets, memory read/writes, and other known computer, processor, and/or process related communication methodologies.

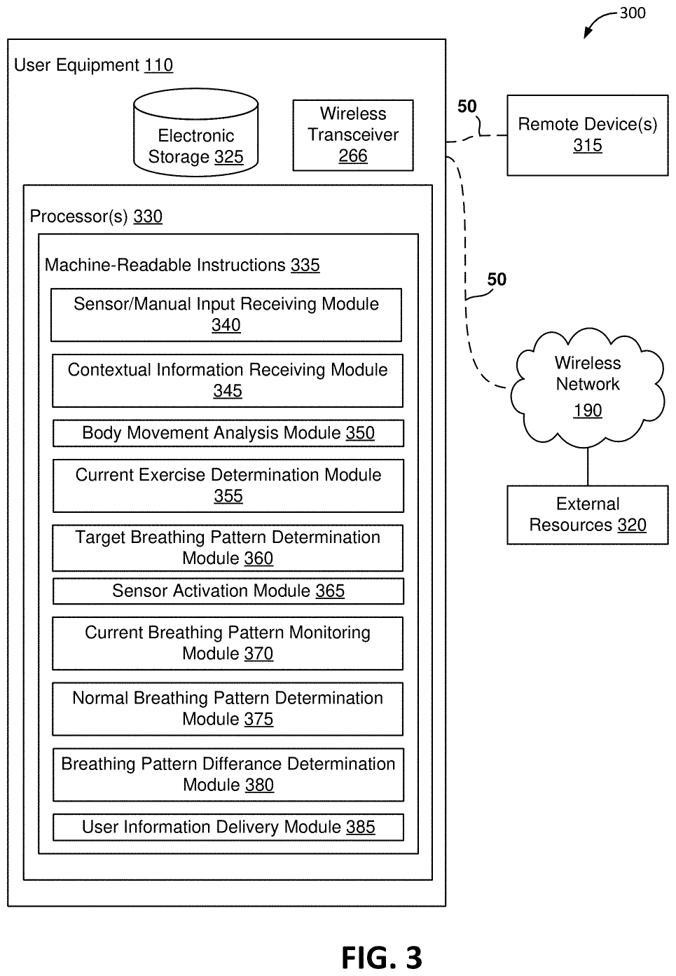

FIG. 3 is a component block diagram illustrating a system 300 configured for providing information regarding breathing patterns of a user executed by a processor of a computing device in accordance with various embodiments. With reference to FIGS. 1A-3, the system 300 may include the user equipment 110 and be configured to communicate with one or more remote device(s) 315 (e.g., 120 130, 140, 150, 160, 170, in FIGS. 1A-1D) or other computing devices via a local wireless connection 50 (e.g., Wi-Fi, Bluetooth, Ant, etc.) or other NFC communication techniques. The user equipment 110 may also be configured to communicate with external resources 320 (e.g., server 195) via a wireless connection 50 to a wireless network 190, such as a cellular wireless communication network.

The user equipment 110 may include electronic storage 325, one or more processors 330, a wireless transceiver 266, and other components. The user equipment 110 may include communication lines, or ports to enable the exchange of information with a network and/or other computing platforms. Illustration of the user equipment 110 in FIG. 3 is not intended to be limiting. The user equipment 110 may include a plurality of hardware, software, and/or firmware components operating together to provide the functionality attributed herein to the user equipment 110.

Electronic storage 325 may include non-transitory storage media that electronically stores information. The electronic storage media of electronic storage 325 may include one or both of system storage that is provided integrally (i.e., substantially non-removable) with the user equipment 110 and/or removable storage that is removably connectable to the user equipment 110 via, for example, a port (e.g., a universal serial bus (USB) port, a firewire port, etc.) or a drive (e.g., a disk drive, etc.). Electronic storage 325 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 325 may include one or more virtual storage resources (e.g., cloud storage, a virtual private network, and/or other virtual storage resources). Electronic storage 325 may store software algorithms, information determined by processor(s) 330, information received from the user equipment 110, information received from remote platform(s) 304, and/or other information that enables the user equipment 110 to function as described herein.

Processor(s) 330 may include one of more processors (e.g., 210, 212, 214, 216, 218, 252, 260), which may be configured to provide information processing capabilities in the user equipment 110. As such, processor(s) 330 may include one or more of a digital processor, an analog processor, a digital circuit designed to process information, an analog circuit designed to process information, a state machine, and/or other mechanisms for electronically processing information. Although processor(s) 330 is shown in FIG. 3 as a single entity, this is for illustrative purposes only. In some embodiments, processor(s) 330 may include a plurality of processing units. These processing units may be physically located within the same device, or processor(s) 330 may represent processing functionality of a plurality of devices operating in coordination.

The user equipment 110 may be configured by machine-readable instructions 335, which may include one or more instruction modules. The instruction modules may include computer program modules. In particular, the instruction modules may include one or more of a sensor/manual input receiving module 340, a contextual information receiving module 345, a body movement analysis module 350, a current exercise determination module 355, a target breathing pattern determination module 360, a sensor activation module 365, a current breathing pattern monitoring module 370, a normal breathing pattern determination module 375, a breathing pattern difference determination module 380, a user information delivery module 385, and/or other instruction modules.

The sensor/manual input receiving module 340 may be configured to receive sensor inputs from one or more sensors (e.g., the adhesive patch sensors 120, smart-glasses 130, smart-watch 140, chest-strap sensor 150, the exercise equipment 160, 170, etc.) communicating information to the user equipment (e.g., 110) and/or remote computing devices 315 within the vicinity of the user equipment 110 (e.g., the smart-glasses 130, smart-watch 140, chest-strap sensor 150, the exercise equipment 160, 170, etc.). The processor may then determine the current exercise performed by the user and/or the target breathing pattern appropriate for the current exercise performed by the user based on the received sensor inputs. The sensors may detect the user performing certain types of actions associated with one or more particular exercises and/or may detect the user's current breathing patterns. By way of a non-limiting example, the sensor information may come from cameras, lidar, light sensors, microphones, IMUs, electromyograms, pressure sensors, and/or proximity/motion sensors. Cameras and lidar may detect movements associated with a particular exercise and/or equipment and/or accessory associated with a particular exercise. Microphones may detect current breathing patterns. IMUs may detect movements associated with a particular exercise. Electromyograms may detect muscle movements and/or activation associated with a particular exercise, as well as muscle movements associated with a current breathing pattern.

In addition, the sensor/manual input receiving module 340 may be configured to receive manual inputs from the user or other operator regarding at least one of the current exercise or the target breathing pattern. For example, the user may manually enter or select an indication as to what exercise is being performed. Similarly, the user may manually enter or select a desired target breathing pattern. In this way, the current exercise and/or the target breathing pattern may be determined based on the received manual input.

The processor(s) 330 of the user equipment may receive sensor information directly from onboard sensors and/or use one or more transceivers (e.g., 256, 266) for detecting available wireless connections 50 (e.g., Wi-Fi, Bluetooth, cellular, etc.) for obtaining sensor information from remote sensors. Also, the sensor/manual input receiving module 340 may be configured to determine whether a detected communication link is available to a wearable or other remote computing device.

The contextual information receiving module 345 may be configured to receive contextual information indicating a context in which the user is performing the current exercise for considering a totality of information available about the environment and/or conditions in which the user is exercising. The determined target breathing pattern may then be further based on the received contextual information. For example, a thermometer may indicate the user is exercising in an extremely cold or hot environment. Similarly, the thermometer may be used to determine the user's body temperature, which if too high may warrant the selection of a lower target breathing pattern for lowering an intensity of the current exercise, which may help the user cool down. In addition, the contextual information may include information about the environment (e.g., humidity, barometric pressure), the time of day, or information about the user's activity levels or wellness that may influence target breathing pattern determinations. Further still, the contextual information may include information from user inputs or other sources, such as the user's age, sex, gender, weight, experience exercising or at least performing the current exercise, and/or current health conditions of the user. In some embodiments, the received contextual information may indicate a context in which the user is performing a current exercise. Thus, the determined target breathing pattern may be further based on the received contextual information.

By way of a non-limiting example, the camera(s) and/or lidar(s) may collect imaging that identifies lighting conditions or other elements of the surrounding environment, thermometers may detect an ambient temperature, the microphones may collect sounds (e.g., a current breathing patterns, coughing, sneezing, etc.), and electromyograms may collect indications of muscle movements (e.g., associated with exercises). Thus, contextual information may be received from sensors that provide information to the user equipment (e.g., 110) and/or local computing devices within the vicinity of the user equipment 110.

In order to determine contextual information, the contextual information receiving module 345 may also access a database, containing one or more data records, stored in memory (e.g., 220, 258, 325) or from a remote source, such as a remote system (e.g., 315) or external resources (e.g., 320) using a transceiver (e.g., 256, 266) and related components. The processor of the user equipment may access the data records to compare previously stored information regarding sensor data with data received from sensors in order to determine what the received sensor indications represent. For example, a lookup table may provide information used to determine the appropriate target breathing pattern that corresponds to current temperature, pressure, and/or humidity conditions.

The body movement analysis module 350 may be configured to determine an extent, type, and/or rate of body movement the user is performing based on the received sensor or manual inputs from the sensor/manual input receiving module 340. The user's body movement may be helpful to detect not only what exercise the user is performing, but more specifically what portion of an exercise the user is performing. Many exercises have multiple parts, each having distinct movements that may be associated with different breathing patterns. In this way, the received user body movement information may indicate which part of an exercise the user is currently performing. In addition, the user's body movement may provide information directly regarding the user's current breathing patterns. The cyclical movement of the user's chest, abdomen, and/or nostrils may generally be correlated to a rate at which the user is breathing or the depth of those breaths. In addition, the way the user is breathing may be determined may be determined based on some sensor inputs, such as whether the user is diaphragmatic breathing versus chest breathing.

In addition, the analysis of the user's body movement may determine what part of the user's body movement may be attributed to the determined current exercise apart from breathing and what part of the user's body movement may be attributed to breathing apart from the movements associated with the current exercise. For example, a user's chest may move up and down as the user runs, jumps, or performs other movements that are naturally part of a given exercise, but such exercise movements may be distinguished from the inhaling or exhaling movements of the user's chest that are a natural part of breathing. By storing historical breathing measurement data, typical chest movements of the user for a determined exercise may be known by the body movement analysis module 350. Thus, the body movement analysis module 350 may compare the user's current chest movements to those typical chest movements to identify improper or unusual breathing patterns.

Users will generally benefit when provided feedback about proper form and breathing during an exercise. For example, proper form for yoga includes not just muscle activity, but especially body alignment and breath, which takes consistent practice to achieve. Similarly, the analysis of body movements by the body movement analysis module 350 may help ensure a user is performing strength training exercises in a safe and effective manner, such as by providing feedback to the user to help the user perform the exercise using only the muscles that should be firing are firing during the completion of a repetition of the exercise. Tracking a user's progress towards optimal alignment over time could be used to increase/maintain the user's motivation to keep practicing regularly. Performing exercises with improper form may sometimes make the exercise easier than it should be or could cause injury to the user. Body alignment may be determined by the body movement analysis module 350 as part of body movement analysis. Further, if muscles that should not be engaged at a certain intensity are being used, then the user would benefit from the body movement analysis module 350 providing real-time feedback as to how to improve form for more effective and safe movements. By way of a non-limiting example, the camera(s) and/or lidar(s) may collect imaging that identifies the body movement, and/or electromyograms may collect indications of muscle movements (e.g., associated with exercises) that may be analyzed by the body movement analysis module 350.

Further still, power during lifting exercises may be determined by the body movement analysis module 350 by measuring the relative speed of a lift across a single rep, set, or workout using IMUs or wearable cameras, such as in smart-glasses (e.g., 130). The speed of the concentric portion of the lift may be used by the body movement analysis module 350 as a measure of power and be used for tracking as well as guidance (in combination with heart rate data) on when to increase weight or repetitions. Similarly, body movement analysis may be used by the body movement analysis module 350 to reduce the weight the user (e.g., 5) is using for an exercise, if power measurements indicate the weight is too high. Lower power may indicate that the user is over-trained and needs to increase rest between workouts or sets. Power measurements by the body movement analysis module 350 may be provided to the user audibly after each rep, allowing the user to apply that information to guide their own performance during an exercise.

By way of a non-limiting example, body movement information may be received by the body movement analysis module 350 from sensors that provide information to the user equipment (e.g., 110) and/or local computing devices within the vicinity of the user equipment 110 (e.g., adhesive patch sensors 120, smart-glasses 130, smart-watch 140, chest-strap sensor 150, the exercise equipment 160, 170, etc.). The body movement analysis module 350 may also analyze body movement information by accessing a database, containing one or more data records, stored in local memory (e.g., 220, 258, 325) or from a remote source, such as a remote system (e.g., 315) or external resources (e.g., 320) using a transceiver (e.g., 256, 266) and related components.

The current exercise determination module 355 may be configured to determine a current exercise performed by the user. In some embodiments, the current exercise may be determined based on the determination from the body movement analysis module 350. In some other embodiments, the current exercise may be determined by current exercise determination module 355 based on a manual user input. In some embodiments, the current exercise may be determined by current exercise determination module 355 based on input from exercise equipment (e.g., 160, 170) dedicated to a particular type of exercise. In some embodiments, the current exercise may be determined by current exercise determination module 355 based on a combination of the body movement analysis, a manual user input, and/or the input from exercise equipment or sensors.

The determination regarding the current exercise may be more reliable after an initial calibration of the current exercise determination module 355 that identifies the exercise. Thus, the user may be prompted to enter, describe, select, and/or perform a particular exercise during calibration of the current exercise determination module 355. The user equipment may process information received from various sensors and calibrate or correlate that information to the indicated exercise during that initial calibration. The sensor data that was gathered during the initial calibration may be used by the current exercise determination module 355 to automatically identify that exercise when later performed by the user again. For example, a processor of the user equipment may record and/or analyze the sensor data received while the user performs a particular exercise and correlate the various sensor readings to that exercise. The calibration process may allow a processor to later correlate similar sensor data to the indicated exercise. Once a calibration process has been completed, the results may be stored in memory in the form of lookup tables, and the processor may use the lookup tables for determining what exercise is being performed.

Alternatively, or additionally, in response to the processor subsequently detecting an exercise recognized from the calibration process, prolonged sensor readings associated with that exercise, once confirmed by the user, may be used to more accurately identify the exercise in the future. The processor may alternatively maintain a rolling average of the sensor reading associated with an exercise and prompt the user when that rolling average changes beyond a threshold. The prompt may ask the user for confirmation of a calculated guess (i.e., an estimate) regarding the exercise being performed to obtain feedback and provide further calibration for generating the update to the previously determined sensor readings associated with the exercise. Various embodiments may apply machine learning to determine updates, which may occur from time to time, and identify user habits or tendencies regarding certain exercises at certain times of day or certain days of the week that may be useful for determining what exercise is performed. The processor may correlate contextual information to the determined update. For example, circumstance in which the user has a temperature, is sweating more than usual, has been unusually active, or when exercise that takes place on a hot or very cold day may be correlated to the determined update for when similar circumstances occur in the future.

The initial calibration of the current exercise determination module 355, may require the user to wear one or more additional sensors that may not be needed to subsequently detect that exercise. By using multiple kinds of sensors during the initial calibration, the processor may subsequently identify the exercise when the user is wearing fewer than all those sensors. In this way, the user may forego wearing all the calibration sensors every time they perform the exercise.

By way of a non-limiting example, the processor(s) 330 of the user equipment may determine the current exercise performed by the user using active and/or passive calibration techniques. Manual entry of an indication of the exercise being performed may be helpful for later identifying that exercise, particularly during the initial calibration process. However, automatic detection of a current exercise by sensors may also or alternatively be used for the initial calibration process to determine baseline parameters that identify the exercise when performed by a particular user. In addition, the baseline parameters may take into account contextual factors that may have influenced detection of the exercise, such as temperature, the environment, the time of day, or information about the user's activity levels, age, sex, weight, or health conditions (e.g., diabetic).

To determine a current exercise, the current exercise determination module 355 may receive information from sensors and/or access a database, containing one or more data records. The records may be stored in a local memory (e.g., 220, 258, 325) or received from a remote source, such as a remote system (e.g., 315) or external resources (e.g., 320) using a transceiver (e.g., 256, 266) and related components. The sensors may similarly be part of the user equipment or received from a remote source, such as a remote system (e.g., 315) or external resources (e.g., 320) using a transceiver (e.g., 256, 266) and related components.

The target breathing pattern determination module 360 may be configured to determine a target breathing pattern appropriate for the current exercise performed by the user. For example, a lookup table may provide information used to determine the appropriate target breathing pattern that corresponds to current exercise performed by the user. The determined target breathing pattern may include a first breathing pattern associated with a first part of the current exercise and a second breathing pattern different from the first breathing pattern and associated with a second part of the current exercise. In some embodiments, more than two different breathing patterns may be associated with an exercise. In some embodiments, the target breathing pattern may be based on a sensor input received from an exercise sensor indicating how the user is moving during an exercise. In some embodiments, the target breathing pattern may be determined based on a context (i.e., contextual information) of the exercise performed (e.g., obtained by the contextual information receiving module 345). Also, in some embodiments, the target breathing pattern may be based on information provided by the user via a user interface, such as the user's body type, health goals, and/or experience level performing the current exercise.

In addition, the processor may receive input from a user indicating that an update is needed for the target breathing pattern. For example, after providing information to the user about the determined target breathing pattern, the user may feel the suggested target breathing pattern is too difficult, not difficult enough, or otherwise needs to be changed. In response to receiving a user input indicating the user wants a change in the target breathing pattern, the processor may recalculate a target breathing pattern, perhaps with further input from the user.

Alternatively, after receiving input from a sensor indicating the current breathing pattern of a user or other biometric reading of the user has exceeded a threshold, the processor may determine and provide the user with a new target breathing pattern that may be safer for the user. For example, a dangerous breathing threshold may be when the user is breathing at a rate under 12 or over 25 breaths per minute for a very mild exercise or higher relative rates for other exercises that demand more exertion. Conditions that may change a user's normal respiratory rate include asthma, anxiety, pneumonia, congestive heart failure, lung disease, use of narcotics, or drug overdose.

By way of a non-limiting example, the processor(s) 330 of the user equipment 110 may determine the target breathing pattern by accessing a database, containing one or more data records, such as a lookup table. The records may be stored in a local memory (e.g., 220, 258, 325) or received from a remote source, such as a remote system (e.g., 315) or external resources (e.g., 320) using a transceiver (e.g., 256, 266) and related components. The records may be maintained on the user equipment or received from a remote source, such as a remote system (e.g., 315) or external resources (e.g., 320) using a transceiver (e.g., 256, 266) and related components.

The sensor activation module 365 may be configured to activate one or more particular sensors in response to certain conditions. In some embodiments, a respiratory sensor configured to determine a current breathing pattern of a user may be activated in response to the processor determining the user is performing an exercise. The additional sensor may be needed due to the nature of the exercise. Alternatively, the additional sensor may be needed so that respiratory sensors may be maintained inactive as much as possible (i.e., when the user is not exercising). In some embodiments, an additional sensor may be activated in response to the current breathing pattern of the user meeting a predetermined threshold. For example, if the user's breathing pattern goes below a low threshold or above a high threshold, the processor may activate an additional sensor to confirm the detected low/high breathing pattern. In some embodiments, a biometric sensor configured to measure a vital sign of the user may be activated in response to the current breathing pattern of the user meeting a predetermined threshold (e.g., a low or high breathing threshold).

By way of a non-limiting example, the additionally activated sensor may include one or more sensors in the user equipment 110 and/or sensors in one or more remote device(s) 315 (e.g., 120 130, 140, 150, 160, 170, in FIGS. 1A-1D).

The current breathing pattern monitoring module 370 may be configured to monitor a current breathing pattern of the user while performing the current exercise based on inputs from a respiratory sensor. The respiratory sensor may be any one or more sensors configured to detect characteristics of a user's breathing, such as the rate, depth, timing, and consistency of breaths of a user. In some embodiments, the current breathing pattern may be monitored by regular or continuous measurements obtained from the body movement analysis module 350. In some embodiments, the current breathing pattern may be determined based on input from exercise equipment (e.g., 160, 170) dedicated to a particular type of exercise or other sensors (e.g., 120, 130, 140, 150). In some embodiments, the current breathing pattern may be determined based a combination of the body movement analysis and/or the input from exercise equipment or other sensors.

Various embodiments include devices that may be configured to measure and track more accurately the breathing pattern of a user by developing, maintaining, and using baseline breathing patterns for an individual under different conditions. For example, controlled breathing pattern measurements may be taken and recorded for an individual, possibly under different circumstances (e.g., varied temperature, time of day, type of activity, etc.), through a calibration or learning process, in which the user breaths normally for a predetermined period of time and the sensors calculate one or more breathing patterns, such as a rate, rhythm, and/or quality of breaths. Such controlled breathing measurements use the active participation and input of the user and is thus referred to herein as “active baselining.” Active baselining may be particularly useful for initially determining the user's regular breathing patterns. The active baselining may also help identify conditions in which the user's breathing is irregular or may be an early warning of a developing health problem. For example, a user breathing heavy after only a low level of physical exertion may be a sign of a respiratory or cardiac problem.

Rather than using a predetermined baseline breathing pattern, a processor implementing various embodiments may compare the currently detected breathing pattern of the user to a target breathing pattern, without considering a baseline breathing pattern. In addition, when a processor receives reassurance that sensor readings accurately measure current breathing patterns (e.g., from redundant sensors), a processor may use that measured breathing pattern to determine, verify, and/or update baseline breathing patterns without the user manually or actively entering information about it, which is herein referred to as “passive baselining.” Passive baselining may be useful for situations in which sensors are reliably able to measure and determine a user's current breathing pattern. In this way, once an active baseline is established for a user, passive baselining may provide a continuous calibration or refinement of the baseline for more accurately estimating the user's breathing pattern.

By way of a non-limiting example, the current breathing pattern of the user may be monitored and determined from the body movement analysis module 350. Also, the current breathing pattern may be determined by information received from sensors that provide information to the user equipment (e.g., 110) and/or local computing devices within the vicinity of the user equipment 110 (e.g., adhesive patch sensors 120, smart-glasses 130, smart-watch 140, chest-strap sensor 150, the exercise equipment 160, 170, etc.).

The current breathing pattern monitoring module 370 may also receive breathing pattern information by accessing a database, containing one or more data records, stored in local memory (e.g., 220, 258, 325) or from a remote source, such as a remote system (e.g., 315) or external resources (e.g., 320) using a transceiver (e.g., 256, 266) and related components. In addition, the current breathing pattern monitoring module 370 may store the determined value(s) of the user's current breathing pattern in local memory (e.g., 220, 258, 325) or that of a remote source, such as a remote system (e.g., 315) or external resources (e.g., 320) using a transceiver (e.g., 256, 266) and related components.

The normal breathing pattern determination module 375 may be configured to determine when the user's current breathing pattern may be within a normal range for the user. Breathing patterns outside the normal range (i.e., abnormal breathing patterns) may be dangerous to the user's health and/or require the initiation of additional operations by the processor. Detection of abnormal breathing patterns may trigger additional information be provided to the user, such as further encouragement or additional instructions regarding breathing or exercise movements. More than slightly abnormal breathing patterns may be a sign of a health risk to the user. For example, the processor may receive inputs indicating the user is breathing at a rate under 12 or over 25 breaths per minute while performing a very mild exercise or higher relative rates for other exercises that demand more exertion. Similarly, a sudden spike in the frequency of a breathing pattern or an erratic breathing pattern may be considered abnormal or even dangerous to the user. Devices implementing some embodiments may be configured to attempt to prevent the user from developing a dangerous breathing pattern by constantly monitoring and encouraging steady breathing appropriate for the user and the current activity (e.g., a particular exercise) or recommending a different cadence or other changes in the movements or breathing associated with the exercise.

By way of a non-limiting example, the processor(s) 330 of the user equipment 110 may determine when the user's current breathing pattern may be considered normal by accessing a database, containing one or more data records, such as a lookup table, indicating what breathing patterns or individual parameters of breathing patterns are dangerous for this particular user. The records may be stored in a local memory (e.g., 220, 258, 325) or received from a remote source, such as a remote system (e.g., 315) or external resources (e.g., 320) using a transceiver (e.g., 256, 266) and related components. The records may be maintained on the user equipment or received from a remote source, such as a remote system (e.g., 315) or external resources (e.g., 320) using a transceiver (e.g., 256, 266) and related components.

The breathing pattern difference determination module 380 may be configured to determine differences between the target breathing pattern determined by the target breathing pattern determination module 360 and the current breathing pattern of the user determined by the current breathing pattern monitoring module 370. In some embodiments determining the differences between the target breathing pattern appropriate for the current exercise performed by the user and the current breathing pattern of the user may comprise comparing the current breathing pattern of the user to at least one of a determined respiratory rate, rhythm, and/or quality of breaths when the user previously performed the current exercise. In some embodiments determining the differences between the target breathing pattern appropriate for the current exercise performed by the user and the current breathing pattern of the user may comprise determining differences between the target breathing pattern appropriate for the first and second parts of the current exercise and the current breathing pattern of the user during the first and second parts of the current exercise.

By way of a non-limiting example, the processor(s) 330 of the user equipment may calculate the differences determined by the breathing pattern difference determination module 380. The differences may be reflected in different rates of breathing, rhythm, or quality of breaths. In addition, the processor(s) 330 of the user equipment may use one or more transceivers (e.g., 256, 266) to transmit the determined differences to a remote computing device (e.g., 120, 130, 140) with instructions regarding what to do with that information.

The user information delivery module 385 may be configured to provide information to the user regarding the determined differences between the target breathing pattern appropriate for the current exercise performed by the user and the current breathing pattern of the user, as determined by the breathing pattern difference determination module 380. In some embodiments, the information provided to the user may include differences between the target breathing pattern appropriate for the first and second parts of the current exercise and the current breathing pattern of the user during the first and second parts of the current exercise. In some embodiments, additional information may be provided to the user regarding another target breathing pattern determined in response to the current breathing pattern exceeding the normal breathing threshold.

The information provided to the user regarding the determined differences between the target and current breathing patterns may include an actual measurement of the rate, volume, and/or steadiness of the user's breathing relative to the target breathing patter. The information provided may be conveyed to the user via a display (e.g., 115) on the user equipment (e.g., 110). Alternatively or additionally, the information provided may be transmitted to a remote source, such as a remote system (e.g., 315) or external resources (e.g., 320) using a transceiver (e.g., 256, 266) and related components. For example, a processor may generate text or render an image on the display of the user equipment to provide an alert message regarding the user's breathing patterns during exercise. In particular, the alert message may tell the user to steady his breath, remind him to synchronize his breathing with certain exercise movements, or remind the user not to hold his breath. Alternatively, the processor may transmit the information to the remote computing device, which is configured to cause the remote computing device to perform the notification function on behalf of the user equipment.

In addition to providing the user with feedback about how or when to perform portions of an exercise, the system may provide a score for how often a users' breath matches designated movements of an exercise. Alternatively, the system may provide a score for how close the user matches a target breathing pattern. The feedback may be provided after the exercise is complete or as a reminder to breathe or how and/or when to breath during a session. Similarly, for weight training, the provided feedback may measure if the user is breathing in on eccentric portion of lift and breathing out on concentric portion of lift and provide the proper feedback.

After receiving input from a sensor indicating the current breathing pattern of a user or other biometric reading of the user has exceeded a threshold, a display and/or audio output from the system may recommend the user change movement patterns, such as a cadence during a run (e.g., change the number of steps between breaths) or bicycle ride (e.g., change the number of pedal revolutions per minute) for determining a more appropriate target breathing pattern. If the user complies with the suggested change in movement patter, but the user's breathing pattern continues to exceed the threshold or exceeds the threshold for a predetermined percentage of the time (e.g., 80%), the system may alert the user to a potential problem and/or advised to rest.

By way of a non-limiting example, the processor(s) 330 of the user equipment 110 may use the display (e.g., 115) and/or a speaker to report the determined breathing pattern differences to the user. In addition, the processor(s) 330 of the user equipment 110 may use one or more transceivers (e.g., 256, 266) to transmit the determined breathing pattern differences to a remote computing device (e.g., 120, 130, 140) with instructions regarding how, when, and/or under what circumstances the remote computing device notification functions to the user may occur.

A remote computing device 315 may include one or more processors configured to execute computer program modules similar to those in the machine-readable instructions 335 described above. By way of a non-limiting examples, in addition to the wearable devices (e.g., 120, 130, 140, 150) described above, the remote computing devices may include one or more of a smart-ring, a smart-appliance, a desktop computer, a laptop computer, a handheld computer, a tablet computing platform, a NetBook, another smart-phone, a gaming console, and/or other computing device.

External resources 320 may include remote servers storing lookup tables in a database (or a backup copy of a lookup database), sources of information outside of system 300, external entities participating with system 300, and/or other resources. In some embodiments, some or all of the functionality attributed herein to external resources 320 may be provided by resources included in system 300.

The processor(s) 330 may be configured to execute modules 340, 345, 350, 355, 360, 365, 370, 375, 380, and/or 385, and/or other modules. Processor(s) 330 may be configured to execute modules 340, 345, 350, 355, 360, 365, 370, 375, 380, and/or 385, and/or other modules by software; hardware; firmware; some combination of software, hardware, and/or firmware; and/or other mechanisms for configuring processing capabilities on processor(s) 330. As used herein, the term “module” may refer to any component or set of components that perform the functionality attributed to the module. This may include one or more physical processors during execution of processor readable instructions, the processor readable instructions, circuitry, hardware, storage media, or any other components.

The description of the functionality provided by the different modules 340, 345, 350, 355, 360, 365, 370, 375, 380, and/or 385 described below is for illustrative purposes, and is not intended to be limiting, as any of modules 340, 345, 350, 355, 360, 365, 370, 375, 380, and/or 385 may provide more or less functionality than is described. For example, one or more of modules 340, 345, 350, 355, 360, 365, 370, 375, 380, and/or 385 may be eliminated, and some or all of its functionality may be provided by other ones of modules 340, 345, 350, 355, 360, 365, 370, 375, 380, and/or 385. As another example, processor(s) 330 may be configured to execute one or more additional modules that may perform some or all of the functionality attributed below to one of modules 340, 345, 350, 355, 360, 365, 370, 375, 380, and/or 385.

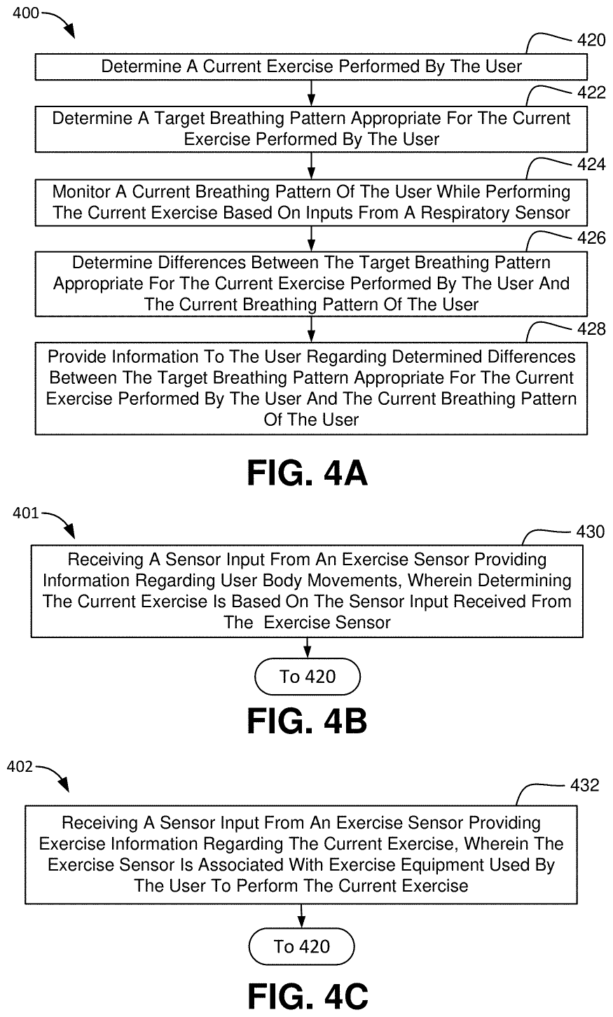

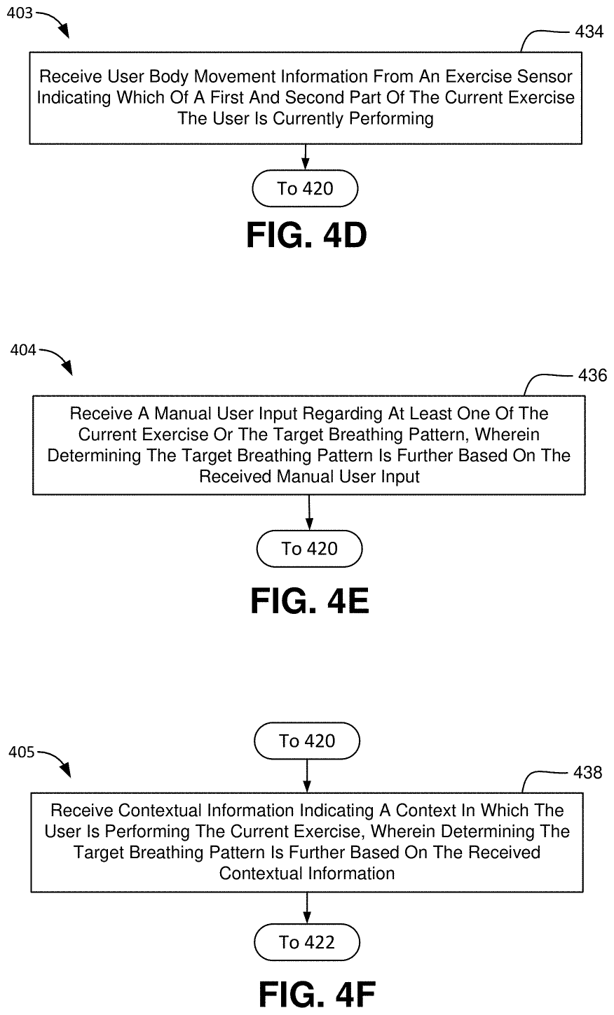

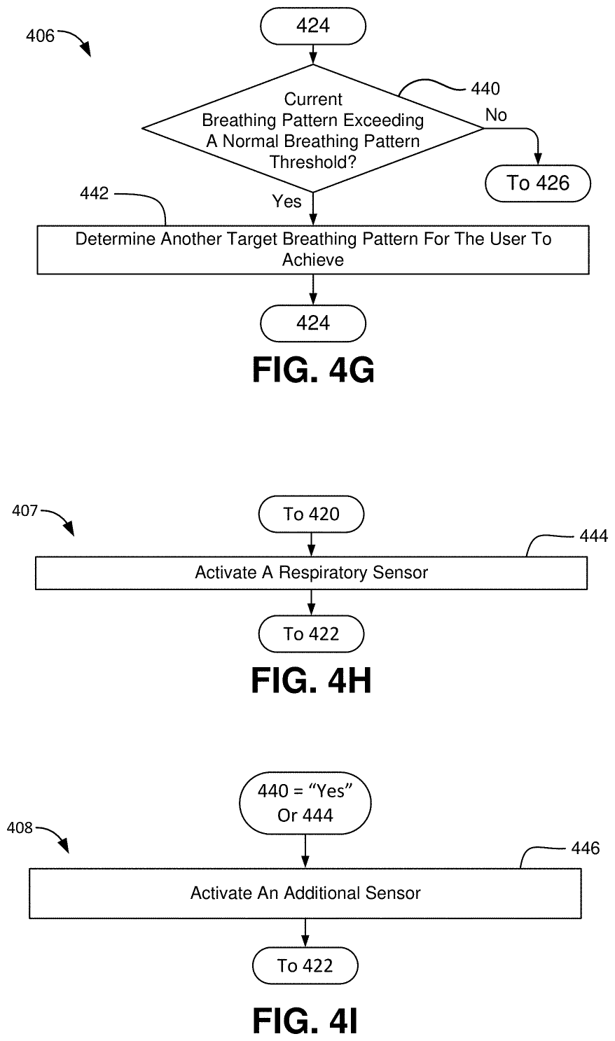

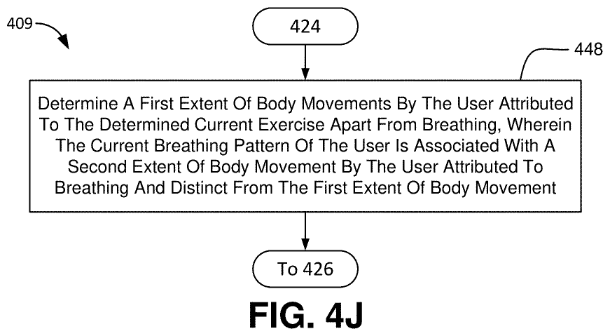

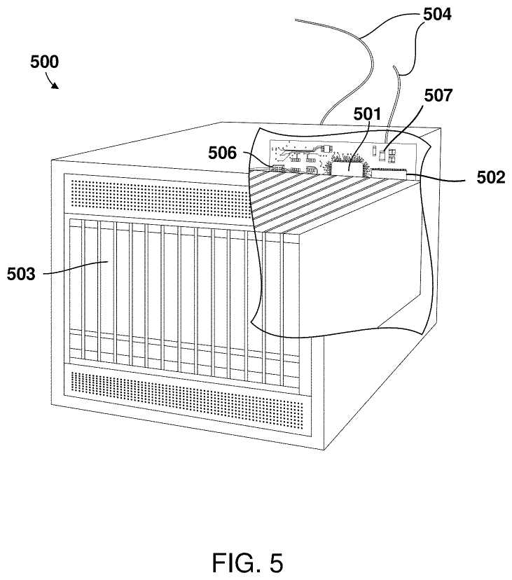

FIG. 4A illustrates a method 400 that may be executed by a processor of user equipment and/or one or more other computing devices of providing respiratory feedback for improved activity performance in accordance with various embodiments. FIGS. 4B, 4C, 4D, 4E, 4F, 4G, 4H, and/or 4I illustrate additional or alternative operations in methods 401, 402, 403, 404, 405, 406, 407, 408, and 409 that may be performed as part of the method 400 in some embodiments. The operations of the methods 400, 401, 402, 403, 404, 405, 406, 407, 408, and 409 are intended to be illustrative. In some embodiments, methods 400, 401, 402, 403, 404, 405, 406, 407, 408, and 409 may be accomplished with one or more additional operations not described, and/or without one or more of the operations discussed. Additionally, the order in which the operations of the methods 400, 401, 402, 403, 404, 405, 406, 407, 408, and 409 are illustrated in FIGS. 4A, 4B, 4C, 4D, 4E, 4F, 4G, 4H, 4I, and 4J and described below is not intended to be limiting.

With reference to FIGS. 1A-J the methods 400, 401, 402, 403, 404, 405, 406, 407, 408, and 409 may be implemented in one or more processors (e.g., 132, 202, 204, 210, 212, 214, 216, 218, 252, 260, 330) of user equipment (e.g., 110) and/or one or more other computing devices, including wearables (e.g., 130, 140, 150) and/or exercise equipment (e.g., 160, 170) configured with processor-executable instructions stored on a non-transitory processor-readable storage medium. The one or more processors may include one or more devices configured through hardware, firmware, and/or software to be specifically designed for execution of one or more of the operations of the methods.

FIG. 4A illustrates a method 400 by which a processor of the user equipment may provide information regarding breathing patterns of a user during exercise in accordance with one or more embodiments.

In block 420, the processor of the user equipment may perform operations including determining a current exercise performed by the user. To make the determination in block 420, the processor may use the body movement analysis module (e.g., 350) and/or the current exercise determination module (e.g., 355). Also, in block 420, the processor may access a database, containing one or more data records, stored in local memory (e.g., 220, 258) or from a remote source, such as a remote system (e.g., 315) or external resources (e.g., 320) using a transceiver (e.g., 256, 266) and related components. The database may provide information regarding known exercises, as well as movements associated with those exercises. Means for performing the operations of block 420 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

In block 422, the processor of the user equipment may perform operations including determining a target breathing pattern appropriate for the current exercise performed by the user. To perform the operations in block 422, the processor may use the target breathing pattern determination module (e.g., 360). Also, in block 422, the processor may access a database, containing one or more data records, stored in local memory (e.g., 220, 258) or from a remote source, such as a remote system (e.g., 315) or external resources (e.g., 320) using a transceiver (e.g., 256, 266) and related components. The database may provide information regarding target breathing patterns, as well as exercises associated with those target breathing patterns. Means for performing the operations of block 422 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

In block 424, the processor of the user equipment may perform operation including monitoring a current breathing pattern of the user while performing the current exercise based on inputs from a respiratory sensor. To perform the operations in block 424, the processor may use the current breathing pattern monitoring module (e.g., 370). Also, in block 424, the processor may receive input about the user's current breathing pattern from sensors included in the user equipment (e.g., 110), devices with sensors (e.g., 120, 130, 140, 150) and/or exercise equipment (e.g., 160, 170). Means for performing the operations of block 424 may include a processor (e.g., 132, 202, 204, 210, 212, 214, 216, 218, 252, 260, 330) coupled to memory (e.g., 220, 258, 325) and one or more devices with sensors (e.g., 110, 120, 130, 140, 150, 160, 170). Other means for performing the operations of block 424 may include a remote source, such as a remote system (e.g., 315) or external resources (e.g., 320) using a transceiver (e.g., 256, 266) and related components.

In block 426, the processor of the user equipment may perform operation including determining differences between the target breathing pattern appropriate for the current exercise performed by the user and the current breathing pattern of the user. To perform the operations in block 426, the processor may use the breathing pattern difference determination module (e.g., 380). Also, in block 426, the processor may access a database, containing one or more data records, stored in local memory (e.g., 220, 258) or from a remote source, such as a remote system (e.g., 315) or external resources (e.g., 320) using a transceiver (e.g., 256, 266) and related components. The database may provide information regarding the target breathing pattern, the current breathing pattern and/or other breathing patterns, as well as exercises associated with those breathing patterns. Means for performing the operations of block 426 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

In block 428, the processor of the user equipment may perform operation including providing information to the user regarding determined differences between the target breathing pattern appropriate for the current exercise performed by the user and the current breathing pattern of the user. To perform the operations in block 428, the processor may use the user information delivery module (e.g., 385). Also, in block 428, the processor may cause a display (e.g., 115) to show information regarding determined differences between the target breathing pattern appropriate for the current exercise performed by the user and the user's current breathing pattern. The information regarding the determined differences may take the form of message to the user (e.g., “Steady Your Breath.”). Additionally, or alternatively, the processor, in block 428, may initiate an audible alert, flash or flicker light, and/or produce a vibration to alert a user. Additionally, or alternatively, the processor may instruct a remote computing device to display an indication of the determined differences in breathing patterns (current versus target). Means for performing the operations of block 428 may include a processor (e.g., 132, 202, 204, 210, 212, 214, 216, 218, 252, 260, 330) coupled to the display (e.g., 115), a speaker, a vibration device, memory (e.g., 220, 258, 325) or from a remote source, such as a remote system (e.g., 315) or external resources (e.g., 320) using a transceiver (e.g., 256, 266) and related components.

Providing information regarding the difference between the user's current breathing pattern and the target breathing pattern may help the user achieve the target breathing pattern and get further benefits from the current exercise. For example, if the processor reminds the user to steady her breathing, she may maximize the benefits of the exercise she performing if she complies.

The operations of the method 400 may repeat, such as if the current exercise changes or the user's current breathing pattern changes.

FIG. 4B illustrates a method 401 in which the processor may provide information regarding breathing patterns of a user during exercise. In block 430, the processor may receive a sensor input from an exercise sensor providing information regarding user body movements, and the determined current exercise in block 420 may be based on the sensor input received from the exercise sensor. For example, the processor may recognize, from motion sensor inputs received from the smart-watch 140, the movement of the user's arms or hands, from a first position (see, position A in FIG. 1A) near the user's chest to a second position (see, position B in FIG. 1A) with arms outstretched away from the chest. Such body movement information may allow the processor to determine the user is performing barbell bench presses (as shown in FIG. 1A). Similarly, images of the bar 92 and weights 94 from camera images received from smart-glasses (e.g., 130) may reinforce the conclusion by the processor that the user is performing barbell bench presses. Similarly, the processor may recognize yoga movements/poses from camera images received from a camera of the user equipment (e.g., 110) and thus determine the user is performing yoga (as shown in FIG. 1B). As another example, the processor receiving sensor inputs from an exercise sensor may recognize a rate and/or type of movement in one or more parts of the body associated with a particular exercise. In this way, cycling movements and/or the cadence thereof, running movements and/or the rate/extent of strides thereof, or swimming movements and/or the rate/extent of strokes thereof may be determined. In some embodiments, the target breathing pattern (e.g., determined in block 422) may be based on the sensor input received from the exercise sensor indicating how the user is moving during the exercise.

To perform the operations in block 430, the processor may use the sensor/manual input receiving module 340. Means for performing the operations of block 430 may include a processor (e.g., 132, 202, 204, 210, 212, 214, 216, 218, 252, 260, 330) coupled to memory (e.g., 220, 258, 325) and a remote source, such as a remote system (e.g., 315) or external resources (e.g., 320) using a transceiver (e.g., 256, 266) and related components.

Following the operations in block 430, the processor may perform the operations in block 420 of the method 400 as described.

FIG. 4C illustrates a method 402 in which the processor may provide information regarding breathing patterns of a user during exercise. In block 432, the processor may receive a sensor input from an exercise sensor providing exercise information regarding the current exercise, wherein the exercise sensor is associated with exercise equipment used by the user to perform the current exercise. For example, the processor may recognize the user running on a treadmill from a message received from the treadmill (e.g., 160) in very close proximity, which indicates the user is running on the treadmill. Alternatively, the processor may receive camera images received from a camera included in the treadmill (e.g., 160) and thus determine the user is running. As another example, the processor may recognize the user exercising on a stationary bicycle (e.g., 170) from a wireless communication link (e.g., 50) between the user equipment (e.g., 110) and the stationary bicycle. Based on the wireless communication link, the processor may determine the user is riding the stationary bicycle. Alternatively, the processor may receive camera images from a camera included in the stationary bicycle (e.g., 170) and thus determine the user is riding a bicycle.

To perform the operations in block 432, the processor may use the sensor/manual input receiving module 340. Means for performing the operations of block 430 may include a processor (e.g., 132, 202, 204, 210, 212, 214, 216, 218, 252, 260, 330) coupled to memory (e.g., 220, 258, 325) and a remote source, such as a remote system (e.g., 315) or external resources (e.g., 320) using a transceiver (e.g., 256, 266) and related components.

Following the operations in block 432, the processor may perform the operations in block 420 of the method 400 as described.

FIG. 4D illustrates a method 403 in which the processor may provide information regarding breathing patterns of a user during exercise. In block 434, the processor may receive a sensor input from an exercise sensor providing exercise information regarding the current exercise, wherein the exercise sensor is associated with exercise equipment used by the user to perform the current exercise. For example, the processor may recognize from camera images received from the smart-glasses 130 or from motion sensor and gyroscopic inputs received from the smart-watch 140 when the users perform different parts of the exercise. Although the overall exercise is barbell bench presses, one part of the exercise may involve the user pressing the barbell away from his chest (e.g., from position A to position B in FIG. 1A). A second part of the exercise may involve the user lowering the barbell from an elevated position to a lower position (e.g., from position B to position A in FIG. 1A). Such body movement information may allow the processor to associate different breathing patterns with different parts of the exercise.

To perform the operations in block 434, the processor may use the sensor/manual input receiving module 340. Means for performing the operations of block 430 may include a processor (e.g., 132, 202, 204, 210, 212, 214, 216, 218, 252, 260, 330) coupled to memory (e.g., 220, 258, 325) and a remote source, such as a remote system (e.g., 315) or external resources (e.g., 320) using a transceiver (e.g., 256, 266) and related components.

Following the operations in block 434, the processor may perform the operations in block 420 of the method 400 as described.

FIG. 4E illustrates a method 404 in which the processor may provide information regarding breathing patterns of a user during exercise. In block 436, the processor may receive a manual user input regarding at least one of the current exercise or the target breathing pattern, wherein determining the target breathing pattern is further based on the received manual user input For example, the processor may receive a manual input entered in a treadmill (e.g., 160) or an exercise bicycle (e.g., 170) indicating what exercise the user is performing. Alternatively, processor may receive a manual input entered in the user equipment (e.g., 110) or a wearable, like a smart-watch (e.g., 140) indicating that the user wants to try to maintain an inefficient target breathing pattern (e.g., 15% faster than an optimal efficiency breathing pattern) while performing the current exercise.

To perform the operations in block 436, the processor may use the sensor/manual input receiving module 340. Means for performing the operations of block 430 may include a processor (e.g., 132, 202, 204, 210, 212, 214, 216, 218, 252, 260, 330) coupled to memory (e.g., 220, 258, 325) and a remote source, such as a remote system (e.g., 315) or external resources (e.g., 320) using a transceiver (e.g., 256, 266) and related components.

Following the operations in block 434, the processor may perform the operations in block 420 of the method 400 as described.

FIG. 4F illustrates a method 405 by which a processor of the user equipment may receive contextual information, which may be used to select the target breathing pattern, in accordance with some embodiments.

In block 438, following the operations in block 420, the processor of the user equipment may perform operations including receiving contextual information indicating a context in which the user is performing the current exercise, wherein determining the target breathing pattern is further based on the received contextual information. For example, the processor may receive a sensor input from a thermometer in the user's smart-watch (e.g., 140), which indicates the user's body temperature is high. In this way, the processor may select a low target breathing pattern to give the user's body a chance to recover from the heat. Alternatively, the processor may receive information from the user equipment (e.g., 110) providing profile and/or biometric information about the user (e.g., user's body type, health goals, experience level performing the current exercise, age, sex, weight, etc.), which may be used to select an appropriate target breathing pattern for a particular exercise. To perform the operations in block 438, the processor may use the contextual information receiving module 345. Means for performing the operations of block 438 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

By way of a non-limiting example, contextual information may be received from sensors that provide information to the user equipment (e.g., 110) and/or local computing devices within the vicinity of the user equipment 110 (e.g., the smart-glasses 130, smart-watch 140, chest-strap sensor 150, etc.). The contextual information may also be received by accessing a database, containing one or more data records, stored in local memory (e.g., 220, 258, 325) or from a remote source, such as a remote system (e.g., 315) or external resources (e.g., 320) using a transceiver (e.g., 256, 266) and related components.

Following the operations in block 438, the processor may perform the operations in block 422 of the method 400 as described.

FIG. 4G illustrates a method 406 in which the processor may determine another target breathing pattern for a user when the user's current breathing pattern is not within a normal range.

In determination block 440, following the operations in block 424, the processor of the user equipment may perform operations including determining whether the current breathing pattern exceeds a normal breathing pattern threshold. For any given person, normal breathing patterns may vary and the individual parameters of that person's breathing patterns may also vary, such as the rate, rhythm, or quality of respiratory movement. Thus, one or more thresholds may be established that represent typical ranges for one or more of those parameters. Those thresholds, if exceeded, may be designed to trigger additional operations by the processor, such as activating one or more additional sensors, determining another target breathing pattern for the user, or providing additional information to the user that may help the user correct the breathing pattern. Individual thresholds may be established for each parameter that may trigger additional operations. In this way, any one of the user's breathing rate, rhythm, or quality of respiratory movement may trigger the system to perform additional operations. In addition, the system may be configured to combine thresholds for each parameter that are lower than the individual thresholds to determine whether to perform additional operations when more than one of such parameters are exceeded. For example, additional operations may be triggered if both the user's combined rate and rhythm threshold are exceeded, even though the higher individual rate and rhythm thresholds are not exceeded. When the user's rate of taking breaths is too high or too low, this may be a sign of the presentation of a life-threatening health issue. The normal breathing pattern threshold may be a predetermined high and/or low limit of breaths per minute. Similarly, unusually shallow breaths (e.g., 25% more shallow than normal for the user), may reflect the development of a dangerous health condition. Thus, a predetermined limit for shallowness of the user's current breathing pattern may be used as the normal breathing pattern threshold. To make the determination in determination block 440, the processor may use the normal breathing pattern determination module (e.g., 375). Also, in determination block 440, the processor may access a database, containing one or more data records, stored in local memory (e.g., 220, 258) or from a remote source, such as a remote system (e.g., 315) or external resources (e.g., 320) using a transceiver (e.g., 256, 266) and related components. The database may provide information about breathing pattern thresholds, beyond which may be considered abnormal or dangerous to a user. Means for performing the operations of block 420 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

In response to determining the user's current breathing pattern does not exceed the normal breathing pattern threshold (i.e., determination block 440=“No”), the processor may perform the operations in block 426.

In response to determining the user's current breathing pattern exceeds the normal breathing pattern threshold (i.e., determination block 440=“Yes”), the processor may perform operations including determining another target breathing pattern for the user to achieve in block 442. To make the determination in block 442, the processor may use the target breathing pattern determination module (e.g., 360). Also, in block 442, the processor may access a database, containing one or more data records, stored in local memory (e.g., 220, 258) or from a remote source, such as a remote system (e.g., 315) or external resources (e.g., 320) using a transceiver (e.g., 256, 266) and related components. The database may provide information regarding target breathing patterns, as well as exercises associated with those target breathing patterns. Means for performing the operations of block 442 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

Following the operations in block 442, the processor may perform the operations in block 424 of the method 400 as described.

FIG. 4H illustrates a method 407 by which a processor of the user equipment may activate a sensor for measuring and/or verifying the user's current breathing pattern, in accordance with some embodiments.

In block 444, following the operations in block 420, the processor of the user equipment may perform operations including activating a respiratory sensor configured to monitor the current breathing pattern of the user while performing the current exercise, in response to determining the current exercise is being performed by the user. The user may be wearing a chest-strap (e.g., 150) that is configured to detect chest movements associated with breathing, but operates in a dormant mode until awakened by a signal from the user equipment (e.g., 110). Thus, in response to the processor of the user equipment determining that the user is exercising, the processor may signal the chest-strap to wake from the dormant mode.

To perform the operations in block 444, the processor may use the sensor activation module 365. Means for performing the operations of block 444 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

Following the operations in block 444, the processor may perform the operations in block 422 of the method 400 as described.

FIG. 4I illustrates a method 408 by which a processor of the user equipment may activate an additional sensor, in accordance with some embodiments.

In block 446, in response to determining the user's current breathing pattern exceeds the normal breathing pattern threshold (i.e., determination block 440=“Yes”), the processor of the user equipment may perform operations including activating an additional sensor. The additional sensor may be another respiratory sensor for redundant and/or more accurate measurements. For example, if one respiratory sensor (e.g., an exercise equipment sensor) is already providing inputs regarding a current breathing pattern, a second respiratory sensor (e.g., on a wearable) may be activated in response to determining the user's current breathing pattern exceeds the normal breathing pattern threshold. Alternatively, the additional sensor may be a biometric sensor for measuring a vital sign of the user. In this way, in response to determining the user's current breathing pattern exceeds the normal breathing pattern threshold of a biometric sensor, like a heart rate monitor, may be activated to ensure the level of exercise does not pose a danger to the user.

Alternatively, following the operations in block 444, the processor of the user equipment may perform operations including activating an additional sensor. For example, in response to the processor determining that the user is performing a high intensity exercise (e.g., in block 420), in addition to activating a respiratory sensor in block 444, in block 446 the processor may active a heart-rate monitor or other biometric sensor, for warning the user in case the user's heart rate or other vital sign gets too high during the exercise. As a further example, in response to the currently active respiratory sensor being determined to be insufficient or not best suited for measuring the breathing patterns of the user, the processor may activate an additional sensor in block 446.

To perform the operations in block 446, the processor may use the sensor activation module 365. Means for performing the operations of block 446 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

Following the operations in block 446, the processor may perform the operations in block 422 of the method 400 as described.

FIG. 4J illustrates a method 409 by which a processor of the user equipment may distinguish body movements associated with the current exercise from body movements associated solely with breathing, in accordance with some embodiments.

In block 448, following the operations in block 424, the processor of the user equipment may perform operations including determining a first extent of body movements by the user attributed to the determined current exercise apart from breathing, wherein the current breathing pattern of the user is associated with a second extent of body movement by the user attributed to breathing and distinct from the first extent of body movement. For example, when performing a barbell bench press exercise (see, FIG. 1A), the user's chest may rise and fall slightly while moving the barbell between a lowered position (e.g., position A) and a raised position (e.g., position B), which chest movement may be associated with a first extent of body movements by the user attributed to the determined current exercise apart from breathing. In addition, the user's chest may rise and fall as part of the normal process of breathing, which chest movement may be attributed to breathing and distinct from the first extent of body movement.

To perform the operations in block 448, the processor may use the body movement analysis module 350 and the current breathing pattern monitoring module 370. Means for performing the operations of block 448 may include a processor (e.g., 132, 202, 204, 210, 212, 214, 216, 218, 252, 260, 330) coupled to memory (e.g., 220, 258, 325) or from a remote source, such as a remote system (e.g., 315) or external resources (e.g., 320) using a transceiver (e.g., 256, 266) and related components.

Following the operations in block 448, the processor may perform the operations in block 426 of the method 400 as described.

Various embodiments (including, but not limited to, embodiments discussed above with reference to FIGS. 1A-4J) may be implemented on a variety of computing devices, an example of which is illustrated in FIG. 5 in the form of a server. With reference to FIGS. 1A-5, the network computing device 500 may include a processor 501 coupled to volatile memory 502 and a large capacity nonvolatile memory, such as a disk drive 503. The network computing device 500 may also include a peripheral memory access device such as a floppy disc drive, compact disc (CD) or digital video disc (DVD) drive 506 coupled to the processor 501. The network computing device 500 may also include network access ports 504 (or interfaces) coupled to the processor 501 for establishing data connections with a network, such as the Internet and/or a local area network coupled to other system computers and servers. The network computing device 500 may include one or more antennas 507 for sending and receiving electromagnetic radiation that may be connected to a wireless communication link. The network computing device 500 may include additional access ports, such as USB, Firewire, Thunderbolt, and the like for coupling to peripherals, external memory, or other devices.

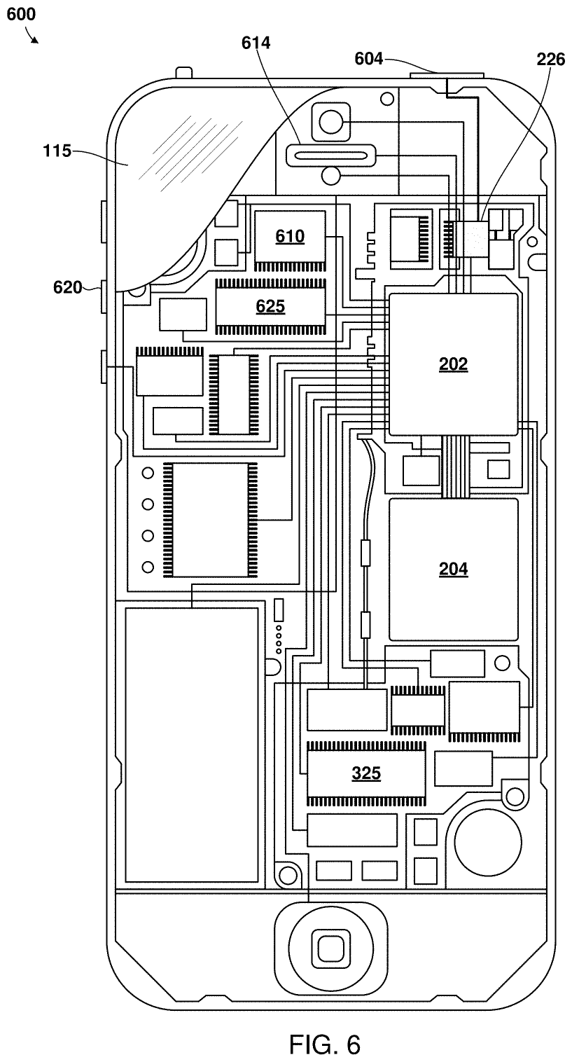

Various embodiments (including, but not limited to, embodiments discussed above with reference to FIGS. 1A-4J) may be implemented on a variety of computing devices, an example of which is illustrated in FIG. 6 in the form of a mobile computing device. With reference to FIGS. 1A-6, a mobile computing device 600 may include a first SoC 202 (e.g., a SoC-CPU) coupled to a second SoC 204 (e.g., a 5G capable SoC), such as D2D links establish in the dedicated ITS 5.9 GHz spectrum communications). The first and/or second SOCs 202, 204 may be coupled to internal memory 325, 625, a display 115, and to a speaker 614. Additionally, the mobile computing device 600 may include one or more antenna 604 for sending and receiving electromagnetic radiation that may be connected to one or more wireless transceivers 266 (e.g., a wireless data link and/or cellular transceiver, etc.) coupled to one or more processors in the first and/or second SOCs 202, 204. Mobile computing devices 600 may also include menu selection buttons or rocker switches 620 for receiving user inputs.

Mobile computing devices 600 may additionally include a sound encoding/decoding (CODEC) circuit 610, which digitizes sound received from a microphone into data packets suitable for wireless transmission and decodes received sound data packets to generate analog signals that are provided to the speaker to generate sound. Also, one or more of the processors in the first and/or second SOCs 202, 204, wireless transceiver 266 and CODEC circuit 610 may include a digital signal processor (DSP) circuit (not shown separately).

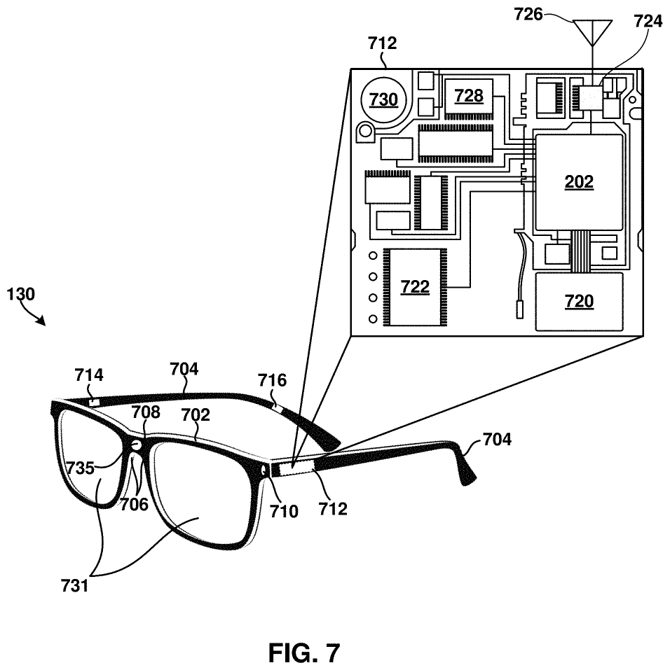

Various embodiments (including embodiments discussed above with reference to FIGS. 1A-4J) may be implemented on a variety of wearable devices, an example of which is illustrated in FIG. 7 in the form of smart glasses 130. With reference to FIGS. 1A-7, the smart glasses 130 may operate like conventional eye glasses, but with enhanced computer features and sensors, like a built-in camera 735 and heads-up display or augmented reality features on or near the lenses 731. Like any glasses, smart glasses may include a frame 702 coupled to temples 704 that fit alongside the head and behind the ears of a wearer. The frame 702 holds the lenses 731 in place before the wearer's eyes when nose pads 706 on the bridge 708 rest on the wearer's nose.

In some embodiments, smart-glasses 130 may include an image rendering device 714 (e.g., an image projector), which may be embedded in one or both temples 704 of the frame 702 and configured to project images onto the optical lenses 731. In some embodiments, the image rendering device 714 may include a light-emitting diode (LED) module, a light tunnel, a homogenizing lens, an optical display, a fold mirror, or other components well known projectors or head-mounted displays. In some embodiments (e.g., those in which the image rendering device 714 is not included or used), the optical lenses 731 may be, or may include, see-through or partially see-through electronic displays. In some embodiments, the optical lenses 731 include image-producing elements, such as see-through Organic Light-Emitting Diode (OLED) display elements or liquid crystal on silicon (LCOS) display elements. In some embodiments, the optical lenses 731 may include independent left-eye and right-eye display elements. In some embodiments, the optical lenses 731 may include or operate as a light guide for delivering light from the display elements to the eyes of a wearer.

The smart-glasses 130 may include a number of external sensors that may be configured to obtain information about wearer actions and external conditions that may be useful for sensing images, sounds, muscle motions and other phenomenon that may be useful for determining an exercise being performed. In some embodiments, smart-glasses 130 may include a camera 735 configured to image objects in front of the wearer in still images or a video stream, which may be transmitted to another computing device (e.g., a mobile device 600) for analysis. In some embodiments, smart-glasses 130 may include a microphone 710 positioned and configured to record sounds in the vicinity of the wearer. In some embodiments, multiple microphones may be positioned in different locations on the frame 702, such as on a distal end of the temples 704 near the jaw, to record sounds emanating from the wearer, such as jaw movements, breathing sounds, and the like. In some embodiments, smart-glasses 130 may include one or more electromyograms 716 mounted on one or both temples 704, such as near the temples or above the ears, and configured to measure electrical activity of the nerves and muscles in the jaw and temple area of a wearer. In some embodiments, smart-glasses 130 may include pressure sensors, such on the nose pads 706, configured to sense facial movements. In some embodiments, smart glasses 130 may include other sensors (e.g., a thermometer, heart rate monitor, body temperature sensor, pulse oximeter etc.) for collecting information pertaining to the user's pulmonary condition, oxygen levels and/or user conditions that may be useful for determining the user's breathing pattern.

The processing system 712 may include a processing and communication SOC 202, which may include one or more processors (e.g., 132, 202, 204, 210, 212, 214, 216, 218), one or more of which may be configured with processor-executable instructions to perform operations of various embodiments. The processing and communication SOC 202 may be coupled to internal sensors 720, internal memory 722, and communication circuitry 724 coupled one or more antenna 726 for establishing a wireless data link with an external computing device (e.g., a mobile device 600), such as via a Bluetooth link. The processing and communication SOC 202 may also be coupled to sensor interface circuitry 728 configured to control and received data from a camera 735, microphone(s) 710, one or more electromyograms 716, and other sensors positioned on the frame 702.

The internal sensors 720 may include an IMU that includes electronic gyroscopes, accelerometers, and a magnetic compass configured to measure movements and orientation of the wearer's head. The internal sensors 720 may further include a magnetometer, an altimeter, an odometer, and an atmospheric pressure sensor, as well as other sensors useful for determining the orientation and motions of the smart glasses 130. Such sensors may be useful in various embodiments for detecting head motions associated with consuming liquids as described.

The processing system 712 may further include a power source such as a rechargeable battery 730 coupled to the SOC 202 as well as the external sensors on the frame 702.

The processors implementing various embodiments may be any programmable microprocessor, microcomputer or multiple processor chip or chips that can be configured by software instructions (applications) to perform a variety of functions, including the functions of the various aspects described in this application. In some communication devices, multiple processors may be provided, such as one processor dedicated to wireless communication functions and one processor dedicated to running other applications. Typically, software applications may be stored in the internal memory before they are accessed and loaded into the processor. The processor may include internal memory sufficient to store the application software instructions.

As used in this application, the terms “component,” “module,” “system,” and the like are intended to include a computer-related entity, such as, but not limited to, hardware, firmware, a combination of hardware and software, software, or software in execution, which are configured to perform particular operations or functions. For example, a component may be, but is not limited to, a process running on a processor, a processor, an object, an executable, a thread of execution, a program, and/or a computer. By way of illustration, both an application running on a processor of a communication device and the communication device may be referred to as a component. One or more components may reside within a process and/or thread of execution and a component may be localized on one processor or core and/or distributed between two or more processors or cores. In addition, these components may execute from various non-transitory computer readable media having various instructions and/or data structures stored thereon. Components may communicate by way of local and/or remote processes, function or procedure calls, electronic signals, data packets, memory read/writes, and other known network, computer, processor, and/or process related communication methodologies.

A number of different cellular and mobile communication services and standards are available or contemplated in the future, all of which may implement and benefit from the various aspects. Such services and standards may include, e.g., third generation partnership project (3GPP), long term evolution (LTE) systems, third generation wireless mobile communication technology (3G), fourth generation wireless mobile communication technology (4G), fifth generation wireless mobile communication technology (5G), global system for mobile communications (GSM), universal mobile telecommunications system (UMTS), 3GSM, general packet radio service (GPRS), code division multiple access (CDMA) systems (e.g., cdmaOne, CDMA1020™), EDGE, advanced mobile phone system (AMPS), digital AMPS (IS-136/TDMA), evolution-data optimized (EV-DO), digital enhanced cordless telecommunications (DECT), Worldwide Interoperability for Microwave Access (WiMAX), wireless local area network (WLAN), Wi-Fi Protected Access I & II (WPA, WPA2), integrated digital enhanced network (iden), C-V2X, V2V, V2P, V2I, and V2N, etc. Each of these technologies involves, for example, the transmission and reception of voice, data, signaling, and/or content messages. It should be understood that any references to terminology and/or technical details related to an individual telecommunication standard or technology are for illustrative purposes only, and are not intended to limit the scope of the claims to a particular communication system or technology unless specifically recited in the claim language.

Implementation examples are described in the following paragraphs. While some of the following implementation examples are described in terms of example methods, further example implementations may include: the example methods discussed in the following paragraphs implemented by a computing device comprising a processor configured with processor-executable instructions to perform operations of the example methods; the example methods discussed in the following paragraphs implemented by a computing device including means for performing functions of the example methods; and the example methods discussed in the following paragraphs implemented as a non-transitory processor-readable storage medium having stored thereon processor-executable instructions configured to cause a processor of a computing device to perform the operations of the example methods.

Example 1. A method executed by a processor of user equipment for providing information regarding breathing patterns of a user during exercise, including: determining a current exercise performed by the user; determining a target breathing pattern appropriate for the current exercise performed by the user; monitoring a current breathing pattern of the user while performing the current exercise based on inputs from a respiratory sensor; determining differences between the target breathing pattern appropriate for the current exercise performed by the user and the current breathing pattern of the user; and providing information to the user regarding determined differences between the target breathing pattern appropriate for the current exercise performed by the user and the current breathing pattern of the user.

Example 2. The method of example 1, further including receiving a sensor input from an exercise sensor providing information regarding user body movements, wherein determining the current exercise is based on the sensor input received from the exercise senor.

Example 3. The method of example 2, wherein the target breathing pattern is based on the sensor input received from the exercise sensor indicating how the user is moving during the exercise.

Example 4. The method of any of examples 1-3, further including receiving a sensor input from an exercise sensor providing exercise information regarding the current exercise, wherein the exercise sensor is associated with exercise equipment used by the user to perform the current exercise.

Example 5. The method of any of examples 1-4, further including receiving user body movement information from an exercise sensor indicating which of a first and a second part of the current exercise the user is currently performing, wherein: the target breathing pattern includes a first breathing pattern associated with the first part of the current exercise and a second breathing pattern different from the first breathing pattern and associated with the second part of the current exercise; determining differences between the target breathing pattern appropriate for the current exercise performed by the user and the current breathing pattern of the user includes determining differences between the target breathing pattern appropriate for the first and second parts of the current exercise and the current breathing pattern of the user during the first and second parts of the current exercise; and providing information to the user includes providing information to the user regarding differences between the target breathing pattern appropriate for the first and second parts of the current exercise and the current breathing pattern of the user during the first and second parts of the current exercise.

Example 6. The method of any of examples 1-5, further including receiving a manual user input regarding at least one of the current exercise or the target breathing pattern, wherein determining the target breathing pattern is further based on the received manual user input.

Example 7. The method of any of examples 1-6, further including receiving contextual information indicating a context in which the user is performing the current exercise, wherein determining the target breathing pattern is further based on the received contextual information.

Example 8. The method of any of examples 1-7, wherein the target breathing pattern is based, at least in part, on at least one of a user's body type, health goals, or experience level performing the current exercise.

Example 9. The method of any of examples 1-8, further including determining another target breathing pattern for the user to achieve in response to the current breathing pattern exceeding a normal breathing pattern threshold; and providing additional information to the user regarding the other target breathing pattern.

Example 10. The method of any of examples 1-9, further including activating the respiratory sensor, configured to monitor the current breathing pattern of the user while performing the current exercise, in response to determining the current exercise is being performed by the user.

Example 11. The method of any of examples 1-10, wherein determining differences between the target breathing pattern appropriate for the current exercise performed by the user and the current breathing pattern of the user includes comparing the current breathing pattern of the user to at least one of a previously determined respiratory rate, rhythm, or quality of the user when the user performed the current exercise.

Example 12. The method of any of examples 1-11, further including activating an additional sensor in response to the current breathing pattern of the user exceeding a normal breathing pattern threshold.

13. The method of any of examples 1-12, further including determining a first extent of body movements by the user attributed to the determined current exercise apart from breathing, wherein the current breathing pattern of the user is associated with a second extent of body movement by the user attributed to breathing and distinct from the first extent of body movements.

Example 14. The method of any of examples 1-13, wherein the current breathing pattern of the user includes at least one of a rate, rhythm, or quality of respiratory movement.

Example 15. The method of any of examples 1-14, wherein providing information to the user regarding the determined differences includes notifying the user through at least one of a visual, audible, or haptic alert.

Example 16. The method of any of examples 1-15, wherein the current exercise is determined based on exercise equipment used by the user and the information regarding the determined differences between the target breathing pattern appropriate for the current exercise performed by the user and the current breathing pattern of the user is provided to the user through feedback from the exercise equipment.

Various aspects illustrated and described are provided merely as examples to illustrate various features of the claims. However, features shown and described with respect to any given aspect are not necessarily limited to the associated aspect and may be used or combined with other aspects that are shown and described. Further, the claims are not intended to be limited by any one example aspect. For example, one or more of the operations of the methods may be substituted for or combined with one or more operations of the methods.

The foregoing method descriptions and the process flow diagrams are provided merely as illustrative examples and are not intended to require or imply that the operations of various aspects must be performed in the order presented. As will be appreciated by one of skill in the art the order of operations in the foregoing aspects may be performed in any order. Words such as “thereafter,” “then,” “next,” etc. are not intended to limit the order of the operations; these words are used to guide the reader through the description of the methods. Further, any reference to claim elements in the singular, for example, using the articles “a,” “an,” or “the” is not to be construed as limiting the element to the singular.

Various illustrative logical blocks, modules, components, circuits, and algorithm operations described in connection with the aspects disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such aspect decisions should not be interpreted as causing a departure from the scope of the claims.

The hardware used to implement various illustrative logics, logical blocks, modules, and circuits described in connection with the aspects disclosed herein may be implemented or performed with a general purpose processor, a digital signal processor (DSP), an ASIC, a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of receiver smart objects, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more aspects, the functions described may be implemented in hardware, software, firmware, or any combination thereof. If implemented in software, the functions may be stored as one or more instructions or code on a non-transitory computer-readable storage medium or non-transitory processor-readable storage medium. The operations of a method or algorithm disclosed herein may be embodied in a processor-executable software module or processor-executable instructions, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include RAM, ROM, EEPROM, FLASH memory, CD-ROM or other optical disk storage, magnetic disk storage or other magnetic storage smart objects, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The preceding description of the disclosed aspects is provided to enable any person skilled in the art to make or use the claims. Various modifications to these aspects will be readily apparent to those skilled in the art, and the generic principles defined herein may be applied to other aspects without departing from the scope of the claims. Thus, the present disclosure is not intended to be limited to the aspects shown herein but is to be accorded the widest scope consistent with the following claims and the principles and novel features disclosed herein.

What is claimed is:

1. A method executed by a processor of user equipment for providing information regarding breathing patterns of a user during exercise, comprising:
determining a current exercise performed by the user;
determining a target breathing pattern appropriate for the current exercise performed by the user;
monitoring a current breathing pattern of the user while performing the current exercise based on inputs from a respiratory sensor;
determining differences between the target breathing pattern appropriate for the current exercise performed by the user and the current breathing pattern of the user; and
providing information to the user regarding determined differences between the target breathing pattern appropriate for the current exercise performed by the user and the current breathing pattern of the user.

2. The method of claim 1, further comprising:
receiving a sensor input from an exercise sensor providing information regarding user body movements, wherein determining the current exercise is based on the sensor input received from the exercise senor.

3. The method of claim 2, wherein the target breathing pattern is based on the sensor input received from the exercise sensor indicating how the user is moving during the exercise.

4. The method of claim 1, further comprising:
receiving a sensor input from an exercise sensor providing exercise information regarding the current exercise, wherein the exercise sensor is associated with exercise equipment used by the user to perform the current exercise.

5. The method of claim 1, further comprising receiving user body movement information from an exercise sensor indicating which of a first and a second part of the current exercise the user is currently performing, wherein:
the target breathing pattern includes a first breathing pattern associated with the first part of the current exercise and a second breathing pattern different from the first breathing pattern and associated with the second part of the current exercise;
determining differences between the target breathing pattern appropriate for the current exercise performed by the user and the current breathing pattern of the user comprises determining differences between the target breathing pattern appropriate for the first and second parts of the current exercise and the current breathing pattern of the user during the first and second parts of the current exercise; and
providing information to the user includes providing information to the user regarding differences between the target breathing pattern appropriate for the first and second parts of the current exercise and the current breathing pattern of the user during the first and second parts of the current exercise.

6. The method of claim 1, further comprising:
receiving a manual user input regarding at least one of the current exercise or the target breathing pattern, wherein determining the target breathing pattern is further based on the received manual user input.

7. The method of claim 1, further comprising:
receiving contextual information indicating a context in which the user is performing the current exercise, wherein determining the target breathing pattern is further based on the received contextual information.

8. The method of claim 1, wherein the target breathing pattern is based on at least one of a user's body type, health goals, or experience level performing the current exercise.

9. The method of claim 1, further comprising:
determining another target breathing pattern for the user to achieve in response to the current breathing pattern exceeding a normal breathing pattern threshold; and
providing additional information to the user regarding the other target breathing pattern.

10. The method of claim 1, further comprising:
activating the respiratory sensor, configured to monitor the current breathing pattern of the user while performing the current exercise, in response to determining the current exercise is being performed by the user.

11. The method of claim 1, wherein determining differences between the target breathing pattern appropriate for the current exercise performed by the user and the current breathing pattern of the user comprises comparing the current breathing pattern of the user to at least one of a previously determined respiratory rate, rhythm, or quality of the user when the user performed the current exercise.

12. The method of claim 1, further comprising:
activating an additional sensor in response to the current breathing pattern of the user exceeding a normal breathing pattern threshold.

13. The method of claim 1, further comprising:
determining a first extent of body movements by the user attributed to the determined current exercise apart from breathing, wherein the current breathing pattern of the user is associated with a second extent of body movement by the user attributed to breathing and distinct from the first extent of body movements.

14. The method of claim 1, wherein the current breathing pattern of the user includes at least one of a rate, rhythm, or quality of respiratory movement.

15. The method of claim 1, wherein providing information to the user regarding the determined differences includes notifying the user through at least one of a visual, audible, or haptic alert.

16. The method of claim 1, wherein the current exercise is determined based on exercise equipment used by the user and the information regarding the determined differences between the target breathing pattern appropriate for the current exercise performed by the user and the current breathing pattern of the user is provided to the user through feedback from the exercise equipment.

17. A user equipment (UE), comprising:
a user interface; and
a processor coupled to the user interface and configured with processor-executable instructions to:
determine a current exercise performed by a user;
determine a target breathing pattern appropriate for the current exercise performed by the user;
monitor a current breathing pattern of the user while performing the current exercise based on inputs from a respiratory sensor;
determine differences between the target breathing pattern appropriate for the current exercise performed by the user and the current breathing pattern of the user; and
provide information to the user through the user interface regarding determined differences between the target breathing pattern appropriate for the current exercise performed by the user and the current breathing pattern of the user.

18. The UE of claim 17, wherein the processor is further configured with processor-executable instructions to:
receive a sensor input from an exercise sensor providing information regarding user body movements; and
determine the current exercise based on the sensor input received from the exercise sensor.

19. The UE of claim 17, wherein the processor is further configured with processor-executable instructions to:
receive a sensor input from an exercise sensor providing exercise information regarding the current exercise, wherein the exercise sensor is associated with exercise equipment used by the user to perform the current exercise.

20. The UE of claim 17, wherein:
the target breathing pattern for the current exercise includes a first breathing pattern associated with the first part of the current exercise and a second breathing pattern different from the first breathing pattern and associated with the second part of the current exercise; and
the processor is further configured with processor-executable instructions to:
receive user body movement information from an exercise sensor indicating which of the first or second part of the current exercise the user is performing;
determine differences between the target breathing pattern appropriate for the current exercise performed by the user and the current breathing pattern of the user by determining differences between the target breathing pattern appropriate for the first and second parts of the current exercise and the current breathing pattern of the user during the first and second parts of the current exercise; and
provide information to the user through the user interface regarding determined differences between the target breathing pattern appropriate for the first and second parts of the current exercise and the current breathing pattern of the user during the first and second parts of the current exercise.

21. The UE of claim 17, wherein the processor is further configured with processor-executable instructions to:
receive a manual user input regarding at least one of the current exercise or the target breathing pattern; and
determine the target breathing pattern further based on the received manual user input.

22. The UE of claim 17, wherein the processor is further configured with processor-executable instructions to:
receive contextual information indicating a context in which the user is performing the current exercise; and
determine the target breathing pattern further based on the received contextual information.

23. The UE of claim 17, wherein the processor is further configured with processor-executable instructions to:
determine another target breathing pattern for the user to achieve in response to the current breathing pattern exceeding a normal breathing pattern threshold; and
provide additional information to the user regarding the other target breathing pattern.

24. The UE of claim 17, wherein the processor is further configured with processor-executable instructions to:
activate the respiratory sensor in response to determining the current exercise is being performed by the user; and
activate an additional sensor in response to the current breathing pattern of the user exceeding a normal breathing pattern threshold.

25. The UE of claim 17, wherein the processor is further configured with processor-executable instructions to:
determine a first extent of body movements by the user attributed to the determined current exercise apart from breathing, wherein the current breathing pattern of the user is associated with a second extent of body movement by the user attributed to breathing and distinct from the first extent of body movements.

26. A non-transitory processor-readable medium having stored thereon processor-executable instructions configured to cause a processor of a user equipment (UE) to provide information regarding breathing patterns of a user during exercise by performing operations comprising:
determining a current exercise performed by the user;
determining a target breathing pattern appropriate for the current exercise performed by the user;
monitoring a current breathing pattern of the user while performing the current exercise based on inputs from a respiratory sensor;
determining differences between the target breathing pattern appropriate for the current exercise performed by the user and the current breathing pattern of the user; and
providing information to the user regarding determined differences between the target breathing pattern appropriate for the current exercise performed by the user and the current breathing pattern of the user.

27. The non-transitory processor-readable medium of claim 26, wherein the stored processor-executable instructions are configured to cause a processor of the UE to perform operations further comprising:
receiving a sensor input from an exercise sensor providing information regarding user body movements, wherein determining the current exercise is based on the received sensor input from the exercise sensor.

28. The non-transitory processor-readable medium of claim 26, wherein:
the stored processor-executable instructions are configured to cause a processor of the UE to perform operations further comprising receiving user body movement information from an exercise sensor indicating which of a first and a second part of the current exercise the user is currently performing; and
the stored processor-executable instructions are configured to cause a processor of the UE to perform operations such that:
the target breathing pattern includes a first breathing pattern associated with the first part of the current exercise and a second breathing pattern different from the first breathing pattern and associated with the second part of the current exercise;
determining differences between the target breathing pattern appropriate for the current exercise performed by the user and the current breathing pattern of the user comprises determining differences between the target breathing pattern appropriate for the first and second parts of the current exercise and the current breathing pattern of the user during the first and second parts of the current exercise; and
providing information to the user comprises providing information to the user regarding differences between the target breathing pattern appropriate for the first and second parts of the current exercise and the current breathing pattern of the user during the first and second parts of the current exercise.

29. The non-transitory processor-readable medium of claim 26, wherein the stored processor-executable instructions are configured to cause a processor of the UE to perform operations further comprising:
receiving contextual information indicating a context in which the user is performing the current exercise; and
determine the target breathing pattern further based on the received contextual information.

30. A user equipment, comprising:
means for determining a current exercise performed by a user;
means for determining a target breathing pattern appropriate for the current exercise performed by the user;
means for monitoring a current breathing pattern of the user while performing the current exercise based on inputs from a respiratory sensor;
means for determining differences between the target breathing pattern appropriate for the current exercise performed by the user and the current breathing pattern of the user; and
means for providing information to the user regarding determined differences between the target breathing pattern appropriate for the current exercise performed by the user and the current breathing pattern of the user.
